(19) United States
(12) Patent Application Publication
(10) Pub. No.: US 2023/0036909 A1
(43) Pub. Date: Feb. 2, 2023

(54) PULMONARY VEIN SHIELD AND METHODS OF USE

(71) Applicant: inQB8 Medical Technologies, LLC, Winchester, MA (US)

(72) Inventors: Arshad Quadri, West Hartford, CT (US); J. Brent Ratz, Winchester, MA (US); Christopher William Stivers, Somerville, MA (US)

(21) Appl. No.: 17/758,068

(22) PCT Filed: Dec. 30, 2020
(86) PCT No.: PCT/US2020/067580

§ 371 Date: Jun. 27, 2022

Related U.S. Application Data
(60) Provisional application No. 62/957,089, filed on Jan. 3, 2020.

(51) Int. Cl. A61M 60/896; A61M 60/174; A61M 60/295

(52) U.S. Cl. CPC A61M 60/896; A61M 60/174; A61M 60/295

(57) ABSTRACT

A system or device for isolating pulmonary pressure from left atrial pressure and/or improving cardiac output. The device may be an implantable cardiac device comprising an intravascular shield. The system may comprise an intravascular shield and a trans-septal delivery sheath. The intravascular shield can be sized and configured to be positioned in a pulmonary vein or a left atrium to restrict fluid flow from the left atrium through one or more pulmonary veins to the lungs while allowing fluid flow from the lungs through the one or more pulmonary veins to the left atrium. The trans-septal delivery sheath can be configured to contain the intravascular shield in a collapsed configuration and deliver the intravascular shield to the left atrium.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### BACKGROUND

### Field

The present disclosure generally relates to implantable cardiac devices and, more particularly, to implantable devices that cover or restrict flow from the left atrium into the pulmonary veins and methods of using the same.

### Description of the Related Art

Heart Failure (HF) is a common problem throughout the world and affects more than 6.5 million people in the United States alone, a number that is expected to increase to nearly 8.5 milling by 2030. While many of these patients are able to live asymptomatically with chronic HF, every year 1.8M patients experience Acute Heart Failure (AHF), a rapid worsening of heart failure symptoms, primarily including dyspnea and fatigue, which requires urgent treatment and immediate hospitalization. In addition to the impact it has on the quality of life for these patients, HF treatments and hospitalizations cost the U.S. healthcare system over $30B annually. AHF is generally split between two classifications, Heart Failure with reduced Ejection Fraction (HFrEF, also referred to as systolic HF) and Heart Failure with preserved Ejection Fraction (HFpEF, also referred to as diastolic HF). While both HFrEF and HFpEF are associated with significant impacts on morbidity and mortality, HFpEF has proven more difficult to address, and despite numerous efforts to develop therapeutic treatments for the disease, diuretics remain one of the only evidence based therapies to placate the effects of HFpEF. As such, in addition to opportunities for improved solutions for HFrEF and Atrial Fibrillation (AF), there is a significant unmet clinical need to develop a meaningful therapeutic solution for patients suffering from HFpEF.

At a certain point in the mechanistic and physiological progression of HF, Left Atrial dysfunction begins to take place. The walls of the Left Atrium (LA) become stiffer and less compliant leading to a reduction in Left Atrial reservoir strain (expansion during filling) and active strain (compression during emptying). This reduction in strain drives increased pressure in the LA which propagates to the lungs (measured by an increase in Pulmonary Capillary Wedge Pressure (PCWP)), reducing lung gas diffusion (measured by diffusion of the lungs for carbon monoxide (DLCO) and arterial and mixed blood gases), which is the fundamental driver of pulmonary congestion and dyspnea, leading to AHF and hospitalization.

In treating HFrEF, the issue resides with the compromised systolic function of the Left Ventricle (LV). As a result, several therapies have been developed to assist the left ventricle in generating systemic pressure and systolic flow to support cardiac output (e.g. LVADs). However, since the systolic function and ejection fraction are preserved with HFpEF, the transference of HFrEF therapies is not well suited or effective.

Research performed in the last several years has highlighted the role of the LA and Left Atrial Pressure in HFpEF. More specifically, research has identified Left Atrial dysfunction (e.g., reduced Left Atrial Reservoir and Active strains) as an independent risk factor associated with HFpEF mortality.

FIG. 1A shows the LA pressure and volume wave forms, which can be combined to depict a “figure-eight” pressure: volume relationship (FIG. 1B).

The expansion of the LA during atrial diastole (through ventricular systole) is known as the reservoir function and is represented by the segments labeled (1) in FIGS. 1A and 1B. Once the mitral valve opens in early diastole, LA and LV pressures equalize and blood passively empties into the LV. This is known as the conduit function and is represented by segment (2) in FIGS. 1A and 1B. Then, at the end of diastole, just before the mitral valve closes, the atrium contracts serving the active pump function represented by segments (4) and (5) in FIGS. 1A and 1B.

In the presence of Congestive Heart Failure (CHF) the normal “figure-eight” illustrated in FIG. 1B is driven up and to the right as LA dilation and volume increase is coupled with increasing stiffness and higher pressures. The increased stiffness also changes the shape of the curves and reduces reservoir strain (reduced expansion during filling) and pump strain (compression during the atrial systole).

While HFpEF is initially associated with increased LV diastolic filling pressures, and the inability to fully evacuate the LA, the resulting fluid backup often results in pulmonary congestion and can translate to pulmonary hypertension, RV-to-PC (Right Ventricle-pulmonary circulation) uncoupling, and right ventricular overload or dysfunction. Consequently, what begins as left-sided heart failure can often progress to right-sided heart failure. Right-sided affects may be observed as an increase in Pulmonary Vascular Resistance (PVR), Pulmonary Artery (PA) systolic pressure (which is equivalent to RV systolic pressure), increased RV workload and inefficiency, and reduced Cardiac Output. Increased LA pressure translates to increased pulmonary artery wedge pressure and increased PVR. This results in increased PA systolic pressure and reduced cardiac output during PA diastole due to a decrease in pressure differential. The increased PA systolic pressure translates to higher workload for the RV during systole and a reduction in efficiency over time.

In addition to being caused by atrial dysfunction, pulmonary hypertension can result from many other causes, all of which contribute to symptoms of dyspnea and fatigue that drive hospitalizations. Mitral regurgitation (MR) is a condition in which blood leaks backwards from the LV to the LA, through the mitral valve (MV). This condition can reduce cardiac output and increase LA pressure, which can ultimately lead to pulmonary hypertension.

In response to the role of elevated LA pressure in exacerbating HFpEF symptoms, intra-atrial devices can be provided that attempt to shunt blood from the LA to the Right Atrium (RA) and thereby reduce LA pressure and PCWP. Early clinic studies have shown promising results, but LA shunting does not fully address congestion in the lungs nor does it help to alleviate the burden on the right side of the heart. Instead, the RA now has to deal with increased volume due to the shunting of the blood from the left side. Furthermore, reducing pressure in the LA alone does not address the underlying atrial stiffness and does not help to restore the complete functionality of the LA in all phases of the cardiac cycle. As an example, reducing LA pressure during the active phase of atrial systole does not generate a larger pressure differential between the LA and the LV. As a result LV End Diastolic filling is not optimized and Cardiac Output is likely to be reduced since volume is being shunted to the right side instead. In addition, LA shunting may not be as effective in patients suffering from Atrial Fibrillation (AF), which is a common condition in HFpEF patients.

### SUMMARY

Some aspects of this disclosure are directed to an implantable cardiac device for isolating pulmonary pressure from left atrial pressure and/or improving cardiac output. The implantable cardiac device can comprise an intravascular shield sized and configured to be positioned in a pulmonary vein or a left atrium, e.g., over one or more ostia of one or more pulmonary veins, to restrict fluid flow from the left atrium through the one or more pulmonary veins to the lungs while allowing fluid flow from the lungs through the one or more pulmonary veins to the left atrium. The implantable cardiac devices as described herein may be suitable for isolating pulmonary pressures, i.e. Pulmonary Vein, Pulmonary Capillary Wedge Pressure (PCWP), from Left Atrial and/or Left Ventricular End Diastolic Pressure, in order to minimize retrograde flow into the pulmonary vein ostia to reduce pulmonary congestion and maximize forward flow into the Left Ventricle to improve cardiac output. In addition to patients suffering from HFrEF, HFpEF, and AF, patients suffering from other disease states may benefit from embodiments of the technology described herein. In particular, the ability of certain embodiments to reduce average PCWP may be beneficial for helping patients with pulmonary hypertension and/or mitral regurgitation (MR). Additionally, patients with both HFpEF and either pulmonary hypertension or MR may particularly benefit from the inclusion of the intravascular shields and other devices as described herein.

In some aspects, the implantable cardiac device of the previous paragraph or any of the implantable cardiac devices described herein may include one or more of the following additional features. The intravascular shield of the implantable cardiac device can comprise a one-way valve sized and configured to be positioned over or within a pulmonary vein. The intravascular shield of the implantable cardiac device can comprise an expandable frame configured to expand within the left atrium over one or more ostia of one or more pulmonary veins. The intravascular shield of the implantable cardiac device can comprise a two or three dimensional shape sized and configured to engage a surface of the left atrium.

The intravascular shield of the implantable cardiac device can comprise an expandable structural element defining a perimeter of the intravascular shield. The perimeter of the intravascular shield can have a shape selected from the group consisting of circular, oval, clover, butterfly, single-lobed, quatrefoil, heart, two-lobed, three-lobed and four-lobed. The intravascular shield of the intravascular cardiac device can comprise a non-porous layer in a center portion and at least one blood regulating flap located around a perimeter that is configured to regulate fluid flow. A perimeter of the intravascular shield can comprise a shape-set wire, a laser cut sheet, or a molded material that is suitable for compression and re-expansion into a catheter.

The intravascular shield of the implantable cardiac device can comprise a plurality of layers. The plurality of layers can comprise a porous layer and a non-porous layer. The non-porous layer can have a plurality of flaps that are configured to open away from the porous layer. The plurality of layers can comprise a woven or knit fabric, a plurality of polymer membranes, a metal mesh, and/or a combination thereof. The porous layer can comprise a plurality of apertures that can align with the plurality of flaps of the non-porous layer. The plurality of apertures can comprise an inner plurality of apertures and an outer plurality of apertures positioned radially outward from the inner plurality of apertures. The plurality of flaps of the valve layer can comprise an inner plurality of flaps and an outer plurality of flaps positioned radially outward from the inner plurality of flaps. The plurality of apertures can comprise a similar shape as the plurality of flaps. The plurality of apertures can comprise smaller dimensions than the plurality of flaps. The non-porous layer can comprise a closed configuration when the plurality of flaps abut the porous layer and an open configuration when the plurality of flaps move away from the porous backing layer. The porous layer can comprise a plurality of holes configured to receive a suture, promote tissue ingrowth, and/or secure the porous layer to the non-porous layer.

The implantable cardiac device can further comprise an elongate delivery device that can have a proximal end and a distal end. The intravascular shield of the implantable cardiac device can be positioned at the distal end of the delivery device.

In another aspect, a system for improving cardiac output is disclosed. The system can comprise the implantable cardiac devices described in any one of the previous paragraphs or any of the implantable cardiac devices described herein and a trans-septal delivery sheath configured to contain the intravascular shield in a collapsed configured and deliver the intravascular shield to the left atrium. The system can further comprise a pressurizing element configured to be positioned in the left atrium. The pressurizing element can be configured to be delivered through the trans-septal delivery sheath to the left atrium. The intravascular shield can be placed distal to the pressurizing element within the trans-septal delivery sheath. The pressurizing element can be a balloon.

In another aspect, a method of improving cardiac output is disclosed. The method can comprise using the implantable cardiac devices or the systems described in any one of the previous paragraphs or described herein.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments.

FIGS. 1A-1B illustrate the five phases in the left atrial pressure-volume relationship.

FIG. 2 illustrates a left atrial cardiac support system according to certain aspects of the present disclosure.

FIG. 3 illustrates a balloon inflation and deflation timeline relative to various portions of the cardiac cycle in accordance with certain aspects of the present disclosure.

FIG. 4 illustrates the change in left atrial pressure with the use of a left atrial balloon relative to various portions of the cardiac cycle.

FIG. 5 illustrates perspective views of a left atrial balloon in various states in accordance with various aspects of the subject technology.

FIG. 6 illustrates partial cross-sectional views of a left atrial balloon in various states in accordance with various aspects of the subject technology.

FIG. 7A-7B illustrate a perspective view and a partial cross-sectional view of a left atrial balloon having a trans-septal shaft and a central lumen in accordance with various aspects of the subject technology.

FIG. 8 illustrates an implanted trans-septal left atrial positioning structure and balloon in accordance with various aspects of the subject technology.

FIG. 9 illustrates an implanted left atrial appendage left atrial positioning structure and balloon in accordance with various aspects of the subject technology.

FIGS. 10A-10D illustrate various views of a left atrial balloon in accordance with various aspect of the subject technology.

FIG. 11 illustrates a schematic of the components of the systems of FIGS. 2 and 13A-13B in accordance with various aspects of the subject technology.

FIG. 12 illustrates a system with which one or more implementations of the subject technology may be implemented.

FIGS. 13A-13B illustrates a dual-sided cardiac support system in inflated and deflated states according to certain aspects of the present disclosure.

FIGS. 14-16 illustrate various states of expansion for a pulmonary artery positioning structure in accordance with various aspects of the subject technology.

FIG. 17 illustrates an implanted pulmonary artery positioning structure in accordance with various aspects of the subject technology.

FIG. 18 illustrates a system having a spiral-shaped right ventricle balloon in accordance with various aspects of the subject technology.

FIG. 19 illustrates an illustration of a heart with a cut-out view of the left atrium and pulmonary veins.

FIG. 20 illustrates a schematic of a left atrium and pulmonary veins of a human heart.

FIGS. 21A-24 illustrates different embodiments of a shield or blood-regulating valve in accordance with various aspects of the subject technology. For example, FIG. 21A illustrates an embodiment of individual one-way valve assemblies implanted in a heart of a patient and FIGS. 21B-21G illustrate a perspective view (FIG. 21B), cross-sectional views (FIGS. 21C and 21E), and bottom views of a closed configuration (FIG. 21F) and an open configuration (FIG. 21G) of the embodiment of the individual one-way valve shown in FIG. 21A in accordance with various aspects of the subject technology.

FIGS. 25A-25B illustrate a bottom view and a cross-sectional view of an embodiment of a shield or blood-regulating valve in accordance with various aspects of the subject technology.

FIGS. 26-28D illustrate different embodiments of a shield or blood-regulating valve in accordance with various aspects of the subject technology.

FIG. 29A illustrates a perspective view of a porcine heart.

FIG. 29B illustrates a perspective view of the porcine heart shown in FIG. 29A with the left atrial appendage and the mitral valve side of the heart removed.

FIG. 29C illustrates a bottom view of the porcine heart shown in FIG. 29B with a transseptal puncture.

FIGS. 30A-30B illustrate an embodiment of a surface shield implanted in the porcine heart shown in FIG. 29C.

FIGS. 31A-31B illustrate the embodiment of the surface shield shown in FIGS. 30A-30B and an embodiment of the left atrial balloon implanted in a porcine heart in accordance with various aspects of the subject technology.

FIG. 32 illustrates an embodiment a frame of a shield in accordance with various aspects of the subject technology.

FIG. 33 illustrates an embodiment of a shield with a frame with a porous backing layer and a mesh layer in accordance with various aspects of the subject technology.

FIGS. 34 -37 illustrate different embodiments of a shield with a non-porous layer containing a plurality of flaps in accordance with various aspects of the subject technology.

FIG. 38 illustrates an embodiments of a three dimensional shield implanted in a porcine heart in accordance with various aspects of the subject technology.

FIGS. 39A-39D illustrate side views (FIGS. 39A and 39C) and top views (FIGS. 39B and 39D) of an embodiment of a three dimensional shield and a left atrial balloon in accordance with various aspects of the subject technology.

FIGS. 40A-40B illustrate a bottom view (FIG. 40A) and a perspective view (FIG. 40B) of an embodiment of a three dimensional shield and a left atrial balloon in accordance with various aspects of the subject technology.

FIGS. 41A-41B illustrate a side view (FIG. 41A) and a perspective view (FIG. 41B) of an embodiment of a support structure for a shield in accordance with various aspects of the subject technology.

FIGS. 42A-42B illustrate a deployed configuration (FIG. 42A) and a partially deployed configuration (FIG. 42B) of an embodiment of a support structure for a shield in accordance with various aspects of the subject technology.

FIGS. 43A-43C illustrate different embodiments of a shield attached to a support structure in accordance with various aspects of the subject technology.

FIGS. 44A-44I illustrate top views (FIGS. 44A, 44D, and 44F), an exploded view (FIG. 44B), perspective views (FIGS. 44C and 44G), a side view (FIG. 44E), and cross-sectional views (FIGS. 44H-44I) of an embodiment of a shield in accordance with various aspects of the subject technology.

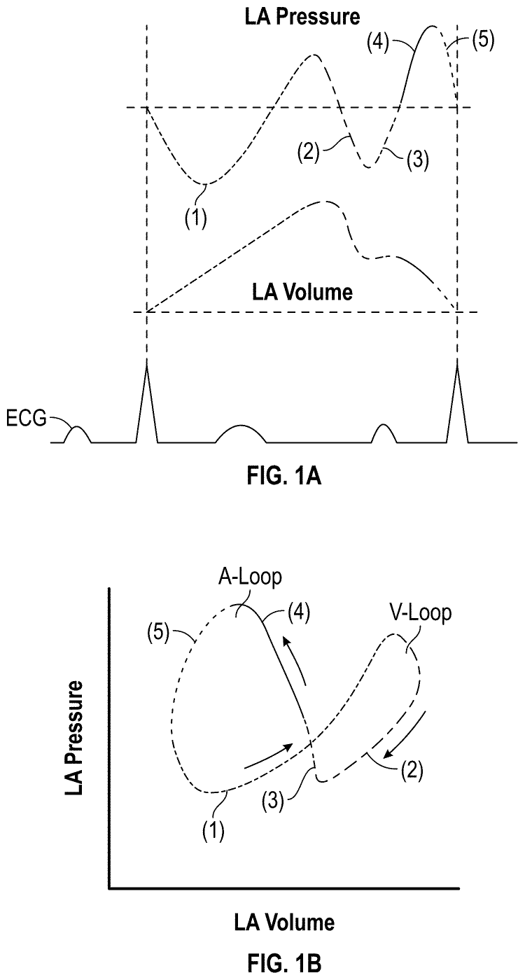

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions may be provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

In the following detailed description, numerous specific details are set forth to provide a full understanding of the present disclosure. It will be apparent, however, to one ordinarily skilled in the art that embodiments of the present disclosure may be practiced without some of the specific details. In other instances, well-known structures and techniques have not been shown in detail so as not to obscure the disclosure.

Aspects of this disclosure are directed to systems and methods for atrial dysfunction, including heart failure and/or atrial fibrillation. It should be appreciated that, although the use of systems such as systems 100, 600 are described below for HF applications, the systems can also be suitable for treatment of non-HF, AF patients based on its ability to restore native LA function and pulsation. Today, it is common to treat AF through ablation procedures, often referred to as maze procedures, whereby the physician uses small incisions, radio waves, freezing, microwave or ultrasound energy to create scar tissue that disrupts the electrical circuitry within the LA in an effort to eliminate the fibrillation. This is often effective via surgery but less effective when done using the currently available interventional techniques. Insufficient ablation could lead to persistent AF while over ablation could lead to scarring that causes the LA walls to stiffen and can ultimately lead to HF. By contrast, using LA balloon 102 as described below to restore LA function (expansion and contraction) could eliminate the symptoms of AF even in the presence of electrical fluctuations and without the need for ablation that could cause excessive scarring. These intra-cardiac support systems and methods for treating atrial dysfunction are further described in U.S. patent application Ser. No. 16/782,997 entitled “Intra-Cardiac Left Atrial and Dual Support Systems,” filed Feb. 5, 2020, and published as U.S. Publication No. US 2020/0246523 A1, which is hereby incorporated by reference in its entirety.

Aspects of the present disclosure are also directed to an intravascular shield designed to prevent or reduce blood flow in a given direction. In some embodiments a shield may serve as a one-way valve to allow flow in one direction while preventing flow in another direction, such as where a native valve does not exist. In other embodiments a shield may reduce the amount and pressure of blood flow in a given direction while allowing for unrestricted flow in another direction.

In the specific case of left-sided heart failure or other conditions with elevated pulmonary capillary wedge pressure and pulmonary congestion, such a shield or one-way valve may be useful when positioned between the primary chamber of the LA and the ostia of one or more pulmonary veins PV. The progression of left-sided heart failure, whether systolic (reduced ejection fraction) or diastolic (preserved ejection fraction), leads to elevated left-atrial pressures, which can then lead to elevated pulmonary capillary wedge pressures, pulmonary congestion, elevated pulmonary artery pressures and a continued retrograde progression towards right-sided heart failure. The placement of a shield between the pulmonary veins PV and the LA may serve to isolate the pulmonary system from elevations in mean left atrial pressure and spikes in left atrial pressure (such as during atrial systole). A shield may be used temporarily or over a longer term, chronic setting with the goal of isolating the pulmonary capillary wedge pressure (PCWP) from left atrial pressure (LAP) and allowing for the relative reduction of PCWP, which should lead to a decrease in pulmonary congestion and an increase in forward flow during atrial systole, resulting in a subsequent increase in cardiac output.

A shield may be used in isolation or with a cardiac support system, which is further described below, or with other systems. For example, the shield may be used in the presence of a counterpulsation balloon placed in the LA, as further described below, or other pressurizing element. In such a case, the presence of the shield can enhance the benefits of the cardiac support system by creating a barrier between the LA and the pulmonary veins PV. Any pressure increase in the LA caused by the counterpulsation system can be directed entirely into the left ventricle, which can reduce PCWP while also increasing cardiac output.

### Left Atrial Cardiac Support System

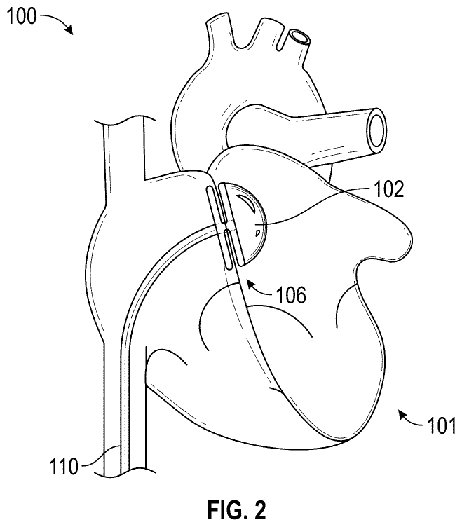

FIG. 2 illustrates an example system 100 in which an implantable pressurizing element has been implanted in the patient. In the example of FIG. 2, system 100 includes a pressurizing element 102 implemented as a balloon for illustrative purposes. As shown, an atrial positioning structure 106 is coupled to the pressurizing element 102 and configured to position the pressurizing element 102 in a LA of a heart 101 of a patient. Although not visible in FIG. 2, system 100 also includes control circuitry configured to operate the pressurizing element 102 to decrease a pressure in the LA during atrial diastole to draw oxygenated blood out of the lungs of the patient by simulating an increase in left atrial reservoir strain and a relative increase in the volume of the LA to reduce a filling pressure in the LA. The control circuitry also operates the pressurizing element 102 to increase the pressure in the LA during atrial systole to simulate an increase left atrial active strain by reducing the relative volume of the LA to increase left atrial pressure during atrial systole. The increase in the left atrial pressure during atrial systole increases a pressure differential between the LA and Left Ventricle that improves diastolic filling of the Left Ventricle.

A feed line 110 is shown, through which a fluid or a gas can be provided or removed for inflation or deflation of balloon implementations of pressurizing element 102, or with which control signals can be provided for operation of other implementations of pressurizing element 102. The feed line 110 may be incorporated into or be part of an elongate catheter body used to deliver the pressurizing element 102 to the LA. For example, in both balloon and non-balloon embodiments, in some aspects a catheter or sheath may be delivered in a percutaneous approach through the femoral vein and advanced through the inferior vena cava, to the Right Atrium RA, and across the atrial septum into the LA. The pressurizing element 102 is positioned at a distal end of the elongate body and may be expanded in the LA. An expandable atrial positioning structure 106, shown proximal to the balloon in FIG. 2, may expand on the left and/or right sides of the septum to help secure the balloon within the LA. In some embodiments, the catheter body carrying the balloon may be delivered through a separate trans-septal sheath that is positioned between the RA and LA.

System 100 may also include one or more sensors such as electrocardiogram (ECG) sensors and/or pressure sensors that generate signals that correspond to portions of the cardiac cycle of the patient. Pressurizing element 102 can be operated to generate pressure changes (e.g., pressure increases and/or pressure decreases) in the LA, in coordination with various portions of the cardiac cycle based on the signals from the sensor.

In accordance with aspects of the present disclosure, the left-atrial support system 100 of FIG. 2 is provided to address potential dysfunction on the left side of the heart, potentially before problems occur on the right side and/or to alleviate dysfunction on both sides of the heart via reducing pulmonary capillary wedge pressure (a proxy for pulmonary congestion) and improved filling of the Left Ventricle.

In contrast with HFpEF treatments with devices that reduce LA pressure only at the cost of increasing the burden on the right side of the heart and decreasing cardiac output, systems 100 as described herein support the heart by reducing the burden on the left side of the heart without adding burden to the right atrium, thereby potentially also reducing congestion and pulmonary wedge pressure and improving LV diastolic filling, which can provide a net increase in cardiac output. This is achieved by placing a fluid/volume displacing system on the left side of the heart (e.g., pressurizing element 102 in the LA). In the example discussed herein in which pressurizing element 102 is implemented as a balloon, the inflation and deflation of the balloon is timed in such a way to optimize support for each patient and keep blood moving in the proper direction at all times during the cardiac cycle.

Deflation of a balloon 102 in the LA during atrial diastole can help draw oxygenated blood out of the lungs by simulating an increase in LA reservoir strain (e.g., increase in volume during filling) and increasing the relative volume of the LA and reducing the filling pressures. Then, by inflating balloon 102 during the active portion of the diastolic cycle (e.g., during atrial systole) the balloon can simulate an increase in pump/active strain by reducing the relative volume in the LA and increasing LA pressure during the active phase of the cycle, thereby increasing the LA-to-LV pressure differential and improving diastolic filling of the Left Ventricle. This operation of LA balloon 102 serves to restore compliance to areas of the heart (e.g., the LA and LV) that are experiencing increased stiffness and wall stress.

In various operational scenarios, balloon 102 (or other implementations of the pressurizing element for fluid/volume displacement in the LA) can be operated depending on the placement of the balloon and the specific needs of each patient.

Inflation and deflation of balloon 102 can be based on an initial (e.g., fixed) timing or can be triggered by sensor signals from electrocardiogram (e.g., EKG or ECG) sensors, pressure sensors (e.g., a pressure sensor in or near the LA), or a combination thereof.

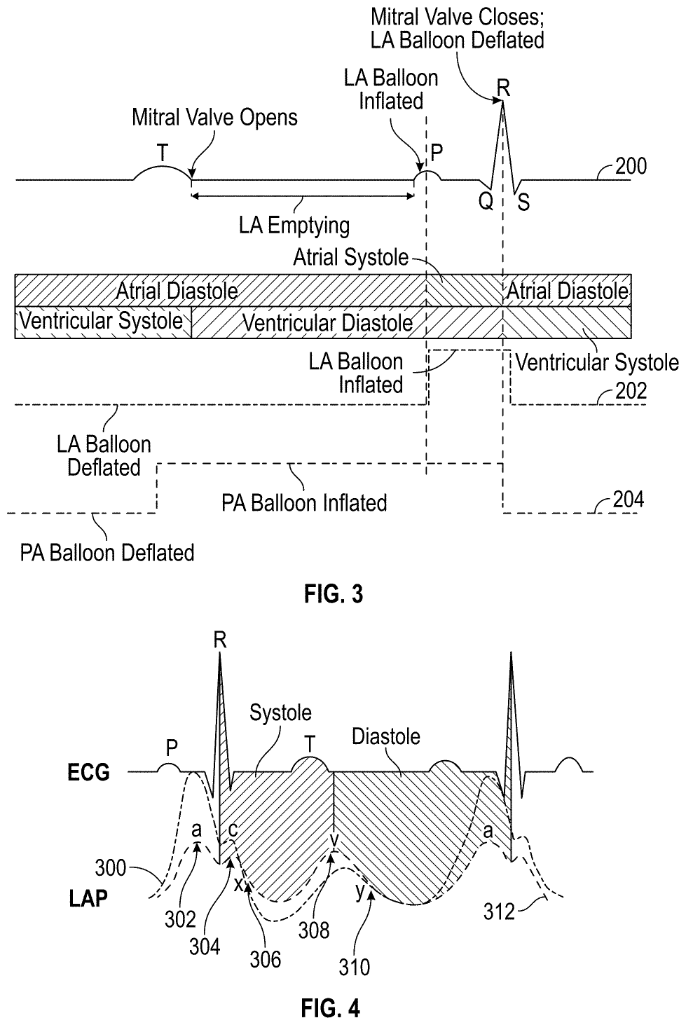

FIG. 3 shows a waveform 202 illustrating a potential sequence of balloon inflations and deflations for the LA balloon 102 against the timing of an ECG signal 200.

In one exemplary implementation of the timing for balloon 102 that can generate the waveforms of FIG. 3, the LA balloon 102 is triggered to deflate upon detection of the R peak plus a time delay (e.g., a 100 millisecond delay after the R peak). In this way, the system initiates deflation of the LA balloon 102 such that deflation of the LA balloon coincides with the natural expansion/reservoir function phase of the LA pressure/volume cycle which occurs during ventricular systole when the mitral valve is closed. LA balloon inflation can be triggered to initiate based on the P wave peak of the ECG or the R peak plus an additional time delay (e.g., a 600 millisecond time delay after the R peak) such that inflation of LA balloon 102 coincides with atrial systole (e.g., with the active contraction portion of the atrial pressure/volume cycle when the a wave peak occurs) at the end of ventricular diastole just before the mitral valve closes to enhance the atrial ventricular pressure differential and increase ventricular filling (e.g., LV End Diastolic Volume, LVEDV).

FIG. 4 shows two LA pressure waveforms 300, 312 against the timing of an ECG signal. The figure also indicates certain points of the cardiac cycle. For example, the LA contracting 302, the mitral valve closing 304, the LA relaxing and filling 306, the LA is full 308, and the LA emptying 310. The unmodified waveform 312 shows a Left atrial pressure waveform for a heart without the use of a LA balloon and modified waveform 300 shows a Left atrial pressure waveform for a heart with the use of a LA balloon. As shown, the a-wave peak (302 equivalent) of the modified waveform 300 is higher than 302 in the unmodified waveform 312 when the LA contracts due to the inflation of the balloon, this wave boost amplifies the natural contractility of the LA which may be diminished as a result of atrial dysfunction related to heart failure and/or atrial fibrillation and serves to improve left ventricular filling and support cardiac output. Conversely, the deflation of the balloon just after the R peak causes a lower pressure during the filling of the atrium (306 equivalent) and a lower v-wave peak (308 equivalent) as compared to the unmodified waveform 312. This reduction in filling pressure should result in a decrease in pulmonary capillary wedge pressure and pulmonary congestion.

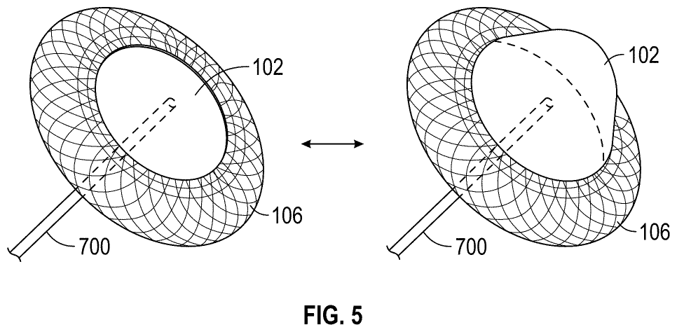

FIGS. 5-9 show exemplary implementations of LA balloon 102 and atrial positioning structure 106.

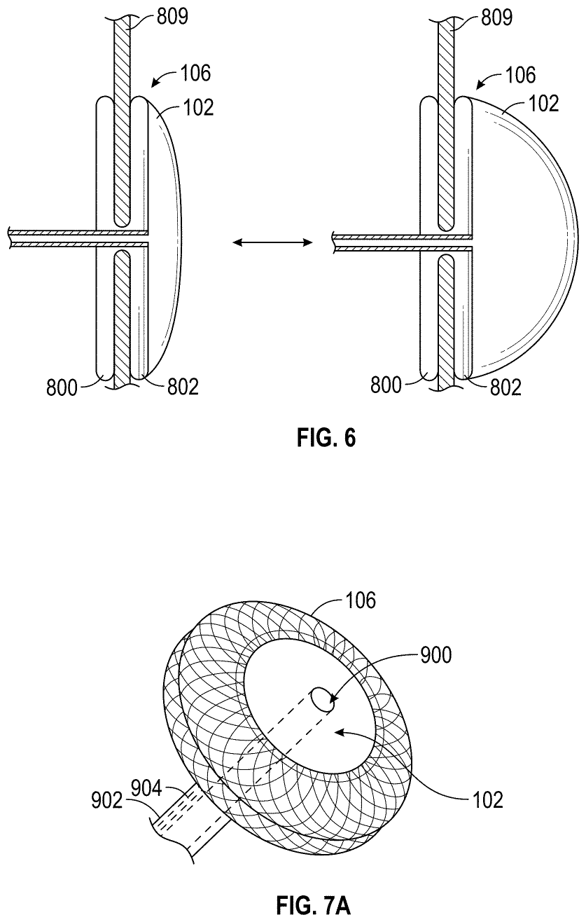

In general, balloon 102 can be separate from its associated positioning structure or can be incorporated with a positioning structure. In either implementation, a positioning structure is provided that maintains the position of its associated balloon within the heart throughout the cardiac cycle. In the example perspective views of FIG. 5, LA balloon 102 is a dome-shaped expandable structure that is attached to atrial positioning structure 106 configured to be positioned trans-septally with a portion 700 that extends through the atrial septum. Portion 700 can also be considered an atrial positioning structure, and may comprise a catheter body as described above that is temporarily positioned within the heart or a shorter trans-septal shaft that may be positioned in the heart over a longer term. The atrial positioning structure 106 comprises a toroidal structure comprising an expandable wire mesh (for example, self-expanding, shape-set nitinol wire mesh) that may substantially take the form of two discs 800 and 802, shown more particularly in FIG. 6. The system may be deployed from within a sheath that can constrain the diameter of the positioning structure 106 and the dome-shaped expandable structure 102 as it is delivered trans-septally. Once the distal end has been advanced into the LA, the sheath may be retracted (or the balloon catheter and positioning structure may be advanced relative to the sheath) such that the distal disc with the balloon 102 attached is able to expand within the LA. The system may then be pulled back towards the Right Atrium to seat the proximal surface of the distal disc against the left atrial facing surface of the septal wall. As the sheath retraction continues, the proximal disc is exposed and expanded such that the distal facing surface of the proximal disc seats against the right atrial facing surface of the septal wall to secure the system relative to the septum. The arrows in FIG. 5 illustrate how balloon 102 can be alternatingly inflated and deflated.

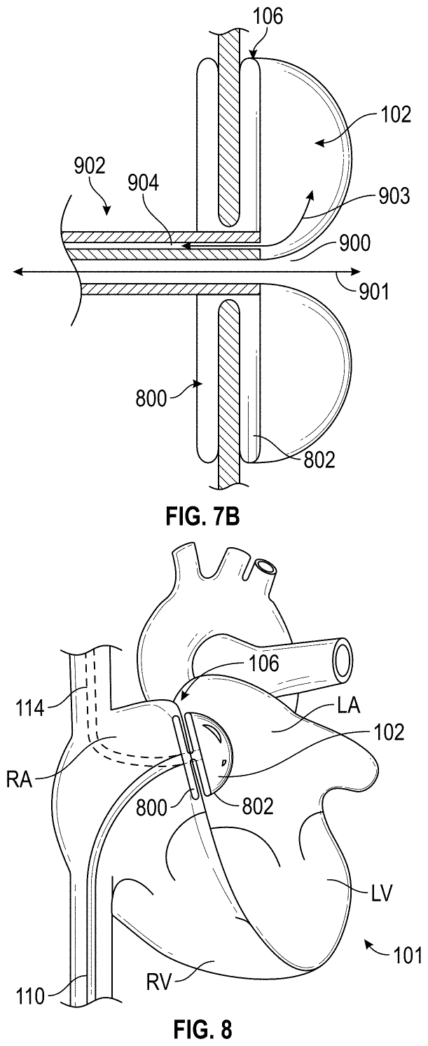

FIG. 6 shows a partial cross-sectional side view of the atrial positioning structure 106 and LA balloon 102 of FIG. 5, anchored with expandable members 800 and 802 on either side of the atrial septum 809 with balloon 102 incorporated into the LA side of the positioning structure 106. Expandable member 800 and 802 can be collapsed for insertion into the patient's heart (and through the atrial septum for member 802) and then expanded to secure positioning structure 106 to the septum. The balloon 102 can comprise anti-thrombotic material. The arrows in FIG. 6 illustrate how balloon 102, once anchored to the septum 809, can be alternatingly inflated and deflated. Although a dome-shaped balloon 106 is shown in FIGS. 6 and 7, it should be appreciated that LA balloon 102 can be shaped as a toroidal loop or other form that allows for trans-septal access to the LA through a central lumen through the balloon 102. The central lumen providing a conduit to the LA can be used as a guidewire lumen to facilitate initial delivery, direct pressure measurement from a hub on the external portion of the catheter, a pressure sensor (e.g. a fiber optic pressure sensor), a shunt path to the venous system, or any other purpose where access to the LA may be desired. For example, FIGS. 7A-7B shows an implementation of LA balloon 102 that comprises a multi-lumen catheter 902 that includes an open central lumen 900 for maintaining access to the left atrial chamber. The catheter 902 includes another lumen 904 that can be used to inflate and deflate the balloon 102. A cross-sectional view of FIG. 7A is shown in FIG. 7B. As shown, the central lumen 900 provide access to the left atrial chamber as shown by the double-headed arrow 901. Additionally, the fluid lumen 904 can deliver fluid to and from the balloon 102 as depicted by the second double-headed arrow. 903.

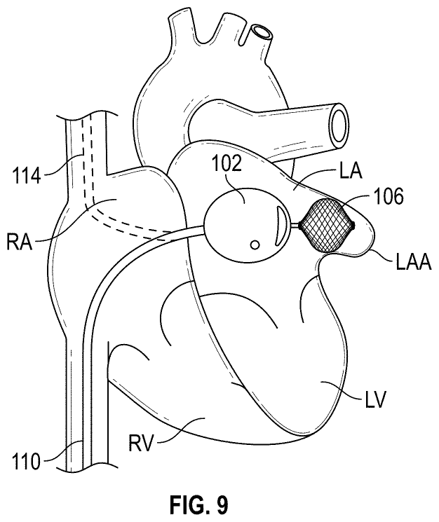

In some operational scenarios, after temporarily treating the patient for HF, a trans-septal LA balloon and atrial anchoring structure can be removed and the trans-septal opening can be closed or left open. FIG. 8 shows LA balloon 102 positioned in the LA by LA positioning structure 106 implemented as a trans-septal anchor having first and second anchor members 800 and 802 respectively disposed in the right and left atria and LA balloon 102 attached to left-side member 802. FIG. 9 shows an alternate implementation in which LA balloon 102 is anchored with a structure 106 that anchors at a distal end in the left atrial appendage (LAA). Anchoring in the LAA (e.g., with an expandable cage as shown in FIG. 9) can also be implemented such that that structure 106 simultaneously closes off a portion of the LAA in order to help reduce overall LA volume and minimize the risk of embolism and/or the effects of AF. It should also be appreciated that LA anchoring structure 106 can be anchored in other locations to position LA balloon 102 in the LA. In one example, LA anchoring structure 106 may be an anchoring member configured for anchoring in an orifice of one or more pulmonary veins. Additionally, in any of the embodiments described herein, and as indicated in FIGS. 8-9, feed line 110 can access the LA from the superior vena cava (SVC), as illustrated by the dotted line 114, or the inferior vena cava (IVC), as illustrated by the solid line 110, via the right atrium.

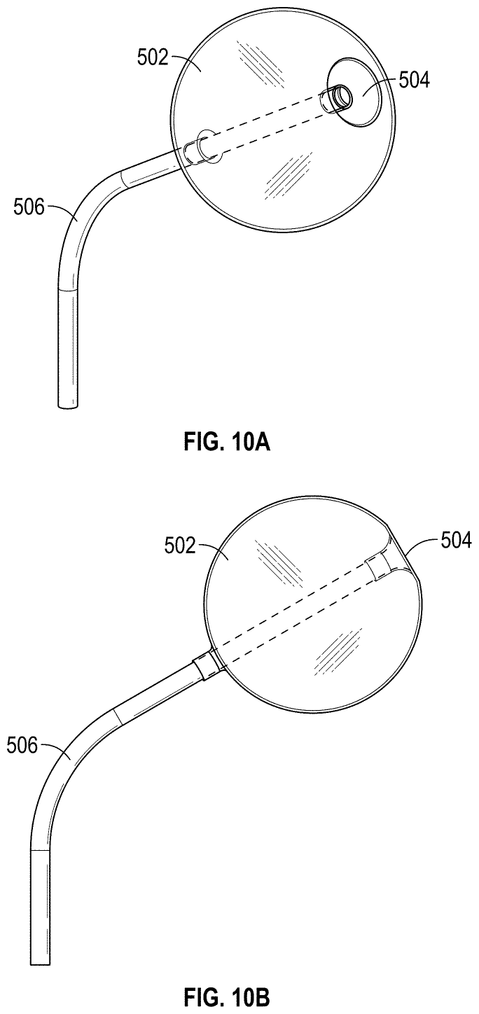

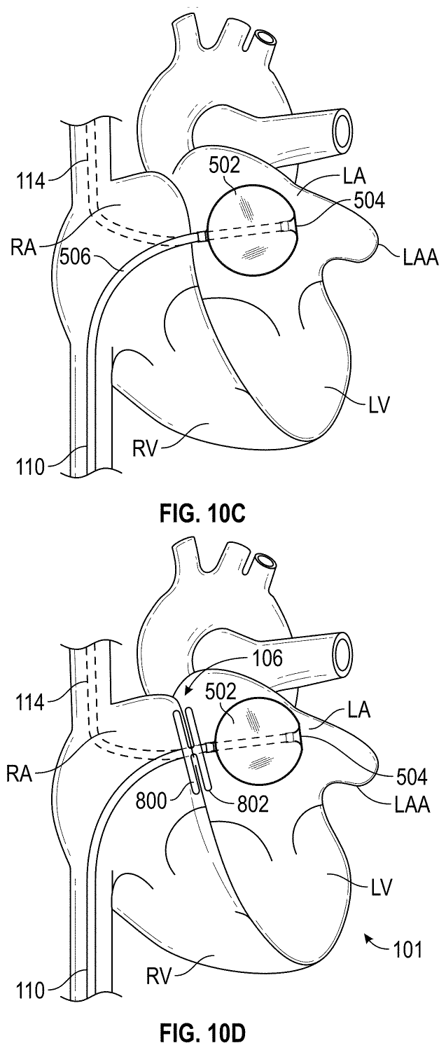

Another implementation of the LA balloon is shown in FIGS. 10A-10D. The distal end 504 of the LA balloon 502 is recessed within the LA balloon 502. The invaginated tip allows for the distal end 504 of the balloon 502 to be atraumatic, including but not limited to instances when a guidewire is not present. The balloon 502 can be anchored to the heart by similar anchoring mechanisms as described above, as shown in FIG. 10D, but it does not require it, as shown in FIG. 10C. FIG. 10C illustrates that the LA balloon 502 can be positioned within the LA using a shaft 506 as the atrial positioning structure. In one implementation, the shaft 506 may be a multi-lumen polymer shaft. The shaft 506 can be pre-formed with a bend or curve of approximately 60 degrees or a variety of different angles to help facilitate proper placement during delivery and stabilization during activation. The shaft 506 can comprise a plurality of lumens. For example, the shaft 506 can have a separate lumen for a guidewire, a separate flow lumen to inflate and deflate the balloon 502, and a separate lumen for a fiber optic pressure sensor. The shaft 506 may also contain a lumen for housing a stiffening stylet for stabilizing the distal tip of the catheter and maintaining balloon position during activation. The stiffening stylet can be inserted before or after the distal tip has been advanced to its desired location. The stiffening stylet may be pre-formed with a bend or curve to impart a desired bend or curve to the shaft 506.

In various implementations, LA balloon 102, 502 can have a shape that is spherical, oval, cylindrical, flat, dome-shaped, toroidal, or any other geometric configuration suitable for pressurizing (e.g., increasing or decreasing pressure in a controllable manner) the LA. The different shapes can improve placement in the patient. In other implementations, the LA balloon 102, 502 can have different sizes to better suit the heart of a patient and/or provide preferential flow patterns upon inflation and/or deflation.

It should also be appreciated that an LA balloon such as LA balloon 102 can be provided in conjunction with one or more other implantable elements.

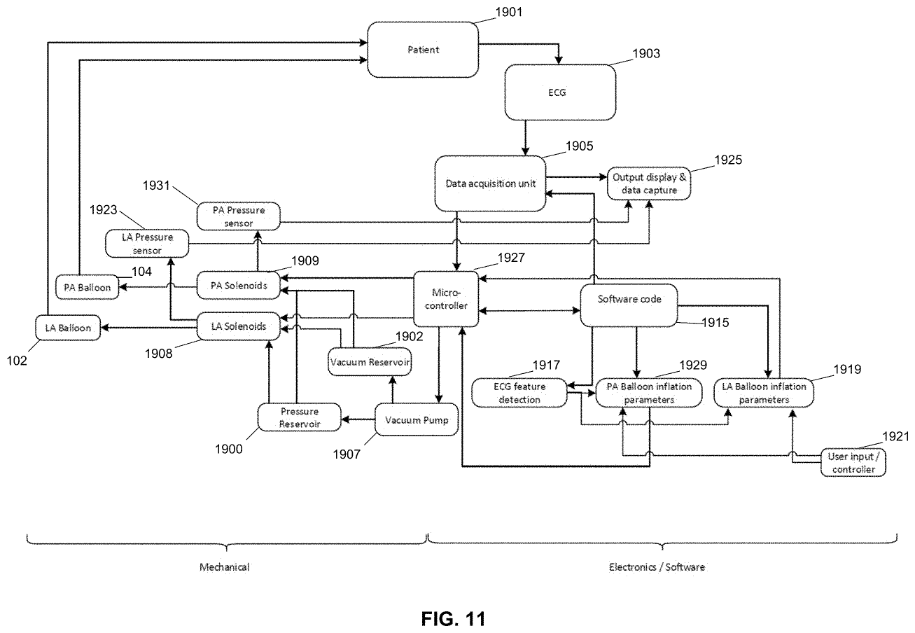

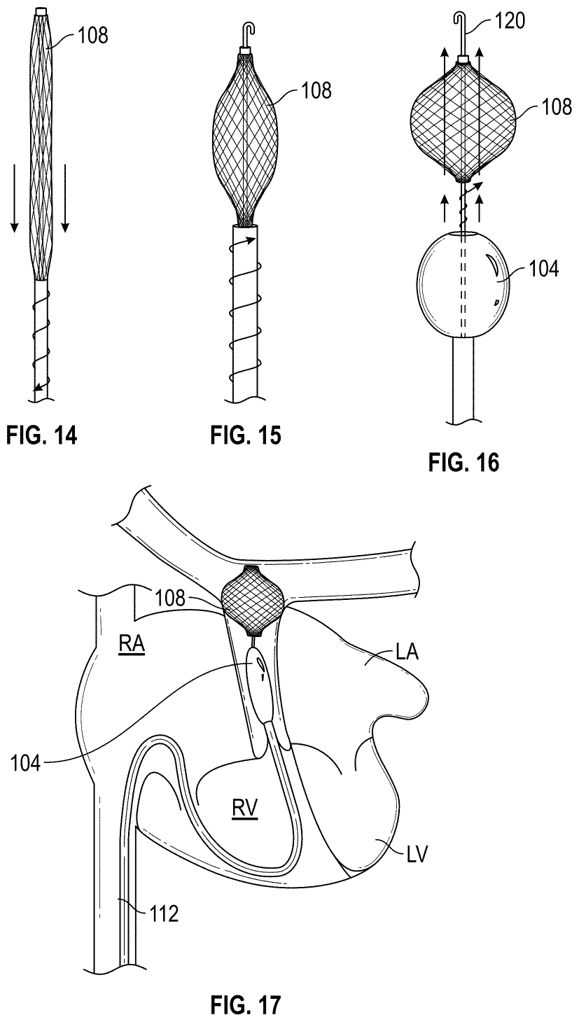

FIG. 11 shows various components that may be incorporated into system 100 described above that are not visible in FIG. 2 and that are configured to operate LA balloon 102 as described herein. FIG. 11 illustrates components that may be usable in a single balloon system, as described above, or in a dual balloon system, as described further below. Therefore, not all of the components illustrated in FIG. 11 may be needed or utilized for a single balloon system. Further details regarding components of the system 100 are also described in U.S. Provisional Application No. 62/801,819, filed February 6, 2019, including but not limited to FIG. 14 and paragraph [0046], the entirety of which is hereby incorporated by reference. In the example of FIG. 11, system 100 may include control circuitry (not shown), a power source (not shown), a pressure chamber or reservoir 1900, a vacuum chamber or reservoir 1902, and a pump 1907. As shown, solenoids 1908 may be disposed on tubing that fluidly couples pressure chamber 1900 and vacuum chamber 1902 to a fluid line (e.g., implementations of fluid line 110 of FIG. 2) can be controlled by control circuitry at microcontroller 1927 to control the inflation and deflations of balloon 102. In one embodiment, ECG sensors 1903 are connected to the patient 1901 and the patient's ECG signal is sent to the data acquisition unit 1905, which is programmed by the software 1915 to look for a set threshold value that correlates to the R-wave in the ECG signal. Once the threshold is detected, the data acquisition unit 1905 sends a pulse (e.g., square wave) to microcontroller 1927. The software 1915 monitors the microcontroller 1927 for the pulses sent by the data acquisition unit 1905 and uses that information to continuously calculate the interval between R-waves (the R-R interval) of the ECG signal. The LA balloon inflation is timed using the calculated R-R intervals and the parameters 1919 (including length of inflation time, offset/delay time after detection of ECG feature 1917, and fill volume), which may be adjusted with the user input/controller 1921. Based on the R-R interval timing and the user input 1921, the software 1915 then communicates with the microcontroller 1927 to actuate the solenoids 1908, opening the balloon lumen(s) to either the pressure chamber 1900 for inflation, or the vacuum chamber 1902 for deflation.

Although system 100 is depicted as an external fixed system (e.g., for bedside support), the components of FIG. 11 and the other figures described above can also be arranged for ambulatory use, or for implantation in the patient (e.g., the drive system for balloon 102 can be in an external console, a wearable external portable unit, or could be fully implantable). System 100 can be provided for temporary, short-term, mid-term, long-term, or permanent use. In temporary cases, LA positioning structure 106 is arranged to be removed from the patient atraumatically.

If desired, balloon 102 can be provided with a pressure sensor/monitor 1923 that collects pressure data within the corresponding cavity, for example a fiber optic pressure sensor or other similar method. Pressure data from this pressure sensor can be used to drive or trigger the balloon inflation and/or deflation and/or can be collected to provide information to the patient, physician, or others in real-time via an output display 1925 or when uploaded separately. In some embodiments, sensors 1923 may also be used to monitor pressure inside the balloon for various purposes.

Although various examples are discussed herein in which LA pressurizing element 102 is implemented as a balloon, it should be appreciated that LA support system 100 can be implemented with other pressurizing elements such as active pumps, axial flow pumps, turbines, or other mechanisms for displacing volume and fluids. More generally, element 102 can be implemented as any suitable combination of pressurizing (e.g., pressure-control), fluid-misplacement, and/or volume-displacement mechanisms that are biocompatible and implantable for positioning in fluid communication with one or more portions of the left side of a patient's heart. For example, LA pressurizing element 102, when operated, may cause a volume displacement in the LA.

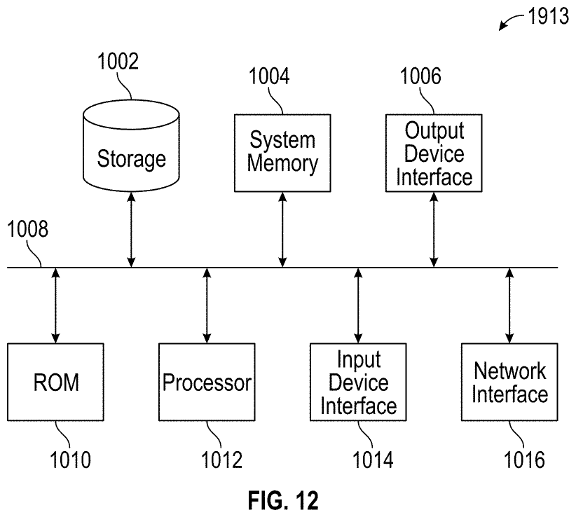

FIG. 12 conceptually illustrates an electronic system with which one or more aspects of the subject technology may be implemented. Electronic system, for example, may be, or may be a part of, control circuitry 1913 for a left atrial support system implemented in standalone device, a portable electronic device such as a laptop computer, a tablet computer, a phone, a wearable device, or a personal digital assistant (PDA), or generally any electronic device that can be communicatively coupled to pressurizing devices implanted in a patient's heart and or pulmonary vasculature. Such an electronic system includes various types of computer readable media and interfaces for various other types of computer readable media. Electronic system includes bus 1008, processing unit(s) 1012, system memory 1004, read-only memory (ROM) 1010, permanent storage device 1002, input device interface 1014, output device interface 1006, and network interface 1016, or subsets and variations thereof.

Bus 1008 collectively represents all system, peripheral, and chipset buses that communicatively connect the numerous internal devices of electronic system. In one or more embodiments, bus 1008 communicatively connects processing unit(s) 1012 with ROM 1010, system memory 1004, and permanent storage device 1002. From these various memory units, processing unit(s) 1012 retrieves instructions to execute and data to process in order to execute the processes of the subject disclosure. The processing unit(s) can be a single processor or a multi-core processor in different embodiments.

ROM 1010 stores static data and instructions that are needed by processing unit(s) 1012 and other modules of the electronic system. Permanent storage device 1002, on the other hand, is a read-and-write memory device. This device is a non-volatile memory unit that stores instructions and data even when electronic system is off. One or more embodiments of the subject disclosure use a mass-storage device (such as a magnetic or optical disk and its corresponding disk drive) as permanent storage device 1002.

Other embodiments use a removable storage device (such as a floppy disk, flash drive, and its corresponding disk drive) as permanent storage device 1002. Like permanent storage device 1002, system memory 1004 is a read-and-write memory device. However, unlike storage device 1002, system memory 1004 is a volatile read-and-write memory, such as random access memory. System memory 1004 stores any of the instructions and data that processing unit(s) 1012 needs at runtime. In one or more embodiments, the processes of the subject disclosure are stored in system memory 1004, permanent storage device 1002, and/or ROM 1010. From these various memory units, processing unit(s) 1012 retrieves instructions to execute and data to process in order to execute the processes of one or more embodiments.

Bus 1008 also connects to input and output device interfaces 1014 and 1006. Input device interface 1014 enables a user to communicate information and select commands to the electronic system and/or a sensor to communicate sensor data to processor 1012. Input devices used with input device interface 1014 include, for example, alphanumeric keyboards, pointing devices (also called “cursor control devices”), cameras or other imaging sensors, electro-cardio sensors, pressure sensors, or generally any device that can receive input. Output device interface 1006 enables, for example, the display of images generated by electronic system. Output devices used with output device interface 1006 include, for example, printers and display devices, such as a liquid crystal display (LCD), a light emitting diode (LED) display, an organic light emitting diode (OLED) display, a flexible display, a flat panel display, a solid state display, a projector, or any other device for outputting information. One or more embodiments may include devices that function as both input and output devices, such as a touch screen. In these embodiments, feedback provided to the user can be any form of sensory feedback, such as visual feedback, auditory feedback, or tactile feedback; and input from the user can be received in any form, including acoustic, speech, or tactile input. Output device interface 1006 may also be used to output control commands for operating pressurizing components (e.g., to control pressurizing element 102) as described herein.

Finally, as shown in FIG. 12, bus 1008 also couples electronic system to a network (not shown) through network interface 1016. In this manner, the computer can be a part of a network of computers (such as a local area network (“LAN”), a wide area network (“WAN”), or an Intranet, or a network of networks, such as the Internet. Any or all components of electronic system can be used in conjunction with the subject disclosure.

### Dual Cardiac Support System

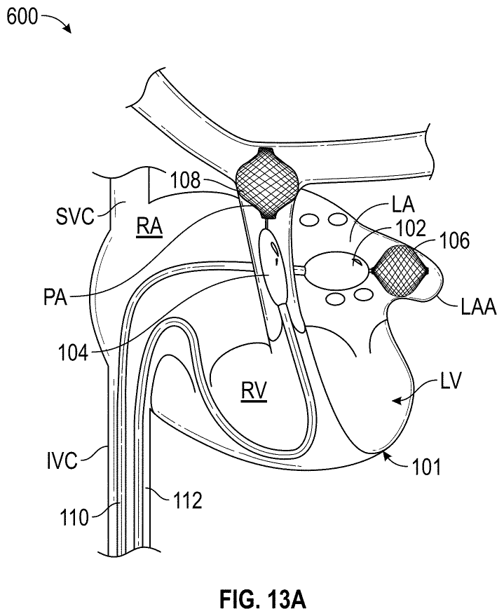

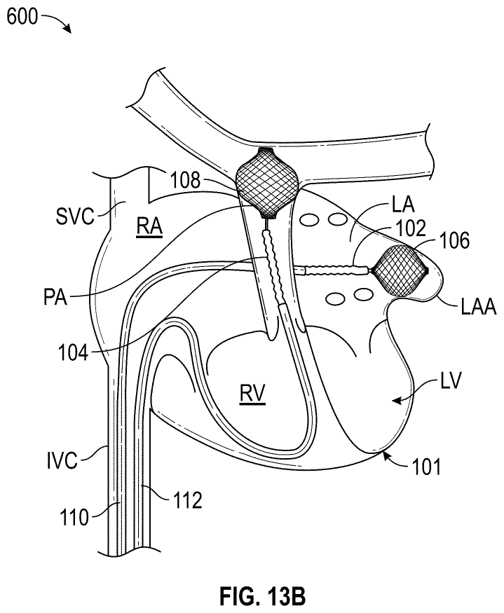

FIGS. 13A-13B illustrate another example system 600 in which two implantable pressurizing elements have been implanted in the patient. In the example of FIGS. 13A-13B, system 600 includes a first pressurizing element 102 implemented as a balloon for illustrative purposes. As shown, an atrial positioning structure 106 is coupled to the first pressurizing element 102 and configured to position the first pressurizing element 102 in a LA of a heart 101 of a patient. Any of the atrial positioning structures described above may be utilized in the system 600 as described herein. As shown, system 600 also includes a second pressurizing element 104 and a pulmonary artery positioning structure 108 coupled to the second pressurizing element 104 and configured to position the second pressurizing element 104 in a Pulmonary Artery PA of the patient. Although not visible in FIGS. 13A-13B, system 600 also includes control circuitry configured to operate the first and second pressurizing elements 102, 104 to generate coordinated pressure modifications and/or volume displacements in the LA and the Pulmonary Artery. Feed lines 110 and 112 are shown, through which a fluid or a gas can be provided or removed for inflation or deflation of balloon implementations of pressurizing elements 102 and 104, or with which control signals can be provided for operation of other implementations of pressurizing elements 102 and 104. As described above, the feed line 110 may be incorporated into or be part of an elongate catheter body used to deliver the pressurizing element 102 to the LA. The feed line 112 may be incorporated into or be part of an elongate catheter used to deliver the pressurizing element 104 and the pulmonary artery positioning structure 108 to the right side of the heart. For example, in both balloon and non-balloon embodiments, in some aspects a catheter or sheath may be delivered in a percutaneous approach through the femoral vein and advanced through the inferior vena cava, to the Right Atrium RA, to the Right Ventricle RV, and into the Pulmonary Artery PA. The pressurizing element 104 is positioned at or near a distal end of the elongate body and may be expanded in the PA. An expandable pulmonary artery positioning structure 108, shown distal to the balloon in FIGS. 13A and 13B, may expand in the Pulmonary Artery (or elsewhere) to help secure the balloon within the PA. In some embodiments, the pulmonary artery positioning structure 108 comprises an expandable cage that may be secured at the bifurcation of the PA.

System 600 may also include one or more sensors such as electrocardiogram (ECG) sensors and/or pressure sensors that generate signals that correspond to portions of the cardiac cycle of the patient. Pressurizing elements 102 and 104 can be operated to generate coordinated pressure changes (e.g., pressure increases and/or pressure decreases) in the LA and Pulmonary Artery respectively, in coordination with various portions of the cardiac cycle based on the signals from the sensor.

In accordance with aspects of the present disclosure, the dual-sided system 600 of FIGS. 13A-13B is provided to address potential dysfunction on both sides of the heart. In contrast with HFpEF treatments with devices that merely reduce LA pressure only at the cost of increasing the burden on the right side of the heart and reducing cardiac output, system 600 as described herein supports the heart by unloading the burden on both side of the lungs, thereby reducing congestion and pulmonary wedge pressure and improving LV diastolic filling to support cardiac output. This is achieved by placing one fluid/volume displacing system on the left side of the heart (e.g., pressurizing element 102 in the LA) and another fluid/volume displacing system on the right side of the heart (e.g., pressurizing element 104 in the Pulmonary Artery). In the example discussed herein in which pressurizing element 102 and pressurizing element 104 are implemented as balloons, the coordinated inflation (see FIG. 13A) and deflation of the balloons (see FIG. 13B) is timed in such a way to optimize support for each patient and keep blood moving in the proper direction at all times during the cardiac cycle. FIG. 13A illustrates when the balloons 102, 104 are inflated and FIG. 13B illustrates when the balloons 102, 104 are deflated.

On the right side, deflation of the balloon can serve to reduce the afterload and work required of the Right Ventricle and improve filling efficiency in the lungs during inflation, as shown in FIG. 13B. For example, actively deflating the PA balloon 104 during PA systole will reduce PA systolic pressures and RV work load. Then subsequently inflating the PA balloon 104, as shown in FIG. 13A, during PA diastole after the pulmonary valve is closed will increase PA diastolic pressure and help overcome pulmonary vascular resistance to provide greater cardiac output. On the left side, deflation of a balloon 102 in the LA during atrial diastole can help draw oxygenated blood out of the lungs by simulating an increase in LA reservoir strain (e.g., increase in volume during filling) increasing the relative volume of the LA and reducing the filling pressures. Then, by inflating balloon 102 during the active portion of the diastolic cycle (e.g., during atrial systole) the balloon can simulate an increase in LA pump/active strain by reducing the relative volume in the LA and increasing LA pressure during the active phase of the cycle, thereby increasing the LA-to-LV pressure differential and improving diastolic filling of the Left Ventricle. This coordinated operation of LA balloon 102 and PA balloon 104 serves to restore compliance to areas of the heart (e.g., the LA and PA) that are experiencing increased stiffness and wall stress.

In various operational scenarios, balloons 102 and 104 (or other implementations of the pressurizing elements for fluid/volume displacement in the LA and PA) can be operated independently or in concert (e.g., with direct synchronicity, exact opposite functionality, or an overlapping sequence with different delays in timing of inflation and deflation throughout the cardiac cycle), depending on the placement of the balloons and the specific needs of each patient.

Inflation and deflation of balloons 102 and 104 can be based on an initial (e.g., fixed) timing or can be triggered by sensor signals from electrocardiogram (e.g., EKG or ECG) sensors, pressure sensors (e.g., a pressure sensor in or near the LA and a pressure sensor in our near the PA), or a combination thereof.

As described above, FIG. 3 shows a waveform 202 illustrating a potential sequence of balloon inflations and deflations for the LA balloon 102 against the timing of an ECG signal 200. FIG. 3 also shows a waveform 204 illustrating a potential sequence of balloon inflations and deflations for the PA balloon 104.

In one exemplary implementation of the timing for balloons 102 and 104 that can generate the waveforms of FIG. 3, the PA balloon 104 is triggered to deflate upon detection of the R peak in the ECG signal and inflate upon detection of the T peak in the ECG signal (or a specific timing offset from the R peak that coincides with the T wave) so that the deflation and inflation coincide with the opening and closing of the pulmonary valve, respectively — and the beginning of systole and diastole respectively. In this example, the LA balloon 102 is triggered to deflate upon detection of the R peak plus a time delay (e.g., a 100 millisecond delay after the R peak). In this way, the system initiates deflation of LA balloon 102 right after initiating the deflation of the PA balloon 104 such that deflation of the LA balloon 102 coincides with the natural expansion/reservoir function phase of the LA pressure/volume cycle which occurs during ventricular systole when the mitral valve is closed. LA balloon 102 inflation can be triggered to initiate based on detection of the peak of the P wave of the ECG or the R peak plus an additional time delay (e.g., a 600 millisecond time delay after the R peak) such that inflation of LA balloon 102 coincides with atrial systole (e.g., with the active contraction portion of the atrial pressure/volume cycle when the a-wave peak occurs) at the end of ventricular diastole just before the mitral valve closes to enhance the atrial ventricular pressure differential and increase ventricular filling (e.g., LV End Diastolic Volume, LVEDV).

FIG. 7 of U.S. Provisional Application No. 62/801,917, filed Feb. 6, 2019, the entirety of which is incorporated by reference herein, shows a series of wave forms that indicate Aortic, PA, Atrial, and Ventricular pressure over the course of two cardiac cycles, against the timing of an ECG signal 1604. In addition, a waveform 1600 illustrating a potential sequence of balloon inflations and deflations for the LA balloon 102 and a waveform 1602 illustrating a potential sequence of balloon inflations and deflations for PA balloon 104 are also shown. In addition, the resulting impact of the balloon inflations of waveforms 1600 and 1602 on the LA and PA pressure waves are illustrated in augmented LA pressure waveform 1606 and augmented PA pressure waveform 1608.

As illustrated in FIGS. 14-17, for PA balloon 104, the PA positioning structure 108 can be distal to the balloon 104 and can be implemented as an expandable cage that anchors against the walls of the PA after expansion from an elongated configuration as shown in FIG. 14 (e.g., for passing through the vascular system to the PA) through an intermediately expanded configuration as shown in FIG. 15, to a fully expanded configuration as shown in FIG. 16 (e.g., rotating a coupled torque shaft counterclockwise could extend the proximal portion from the distal portion along an internal thread and compress the anchoring structure, while rotating the torque shaft clockwise could bring the distal and proximal ends of the anchoring structure closer together and expand its diameter). FIG. 16 also shows PA balloon 104 in an inflated configuration. Also shown in FIGS. 14-16 is a guidewire 120 that can be independently inserted and advanced into the desired location within the anatomy (in this case the PA) before the balloon catheter and anchoring system are introduced, such that the balloon catheter and anchoring system can be tracked into position over the guidewire. The guidewire can then be removed or left in place during the course of treatment.

Although FIGS. 14-17 show PA positioning structure 108 distally disposed relative to PA balloon 104, it should be appreciated that PA positioning structure 108 can be disposed proximal to PA balloon 104 or incorporated in-line with the balloon (e.g., as a cage around the balloon). As indicated in FIG. 16, PA positioning structure 108 allows blood flow therethrough.

FIG. 17 shows PA balloon 104 positioned within the PA by PA positioning structure 108 implemented as an expanded cage at the top of the PA. As indicated in FIG. 17, feed line 112 can access the PA from the SVC or inferior IVC, as illustrated by the solid line 112, via the right atrium and right ventricle.

In various implementations, LA balloon 102 and PA balloon 104 can have the same shape or different shapes, with the shape of either balloon being spherical, oval, cylindrical, flat, dome-shaped, toroidal, or any other geometric configuration suitable for pressurizing (e.g., increasing or decreasing pressure in a controllable manner) the LA and/or the PA.

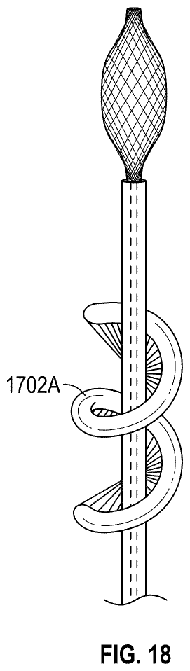

Although HFpEF treatments using a system 100 having a LA pressurizing element 102 and a PA pressurizing element 104 are described herein, other systems for treatment of HFpEF and/or AF are contemplated herein that address the dual-sided problem in accordance with the cardiac cycle features discussed in connection with FIG. 2. As another example, FIG. 18 illustrates a balloon 1702A that is shaped as a spiral to enhance forward flow boost. The balloon 1702A may be configured for positioning in the PA as described above. In other embodiments, any of the balloons or pressurizing elements as described herein in the PA may be configured for positioning in the Right Ventricle RV.

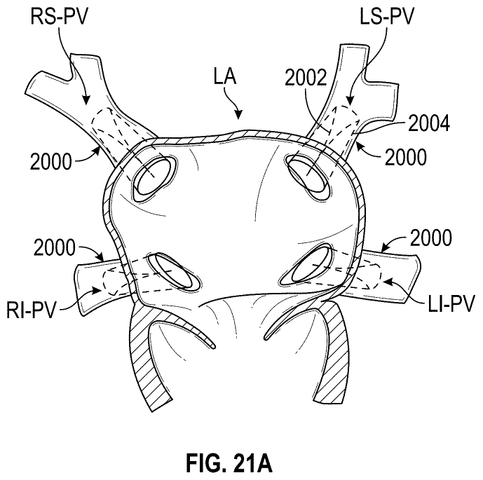

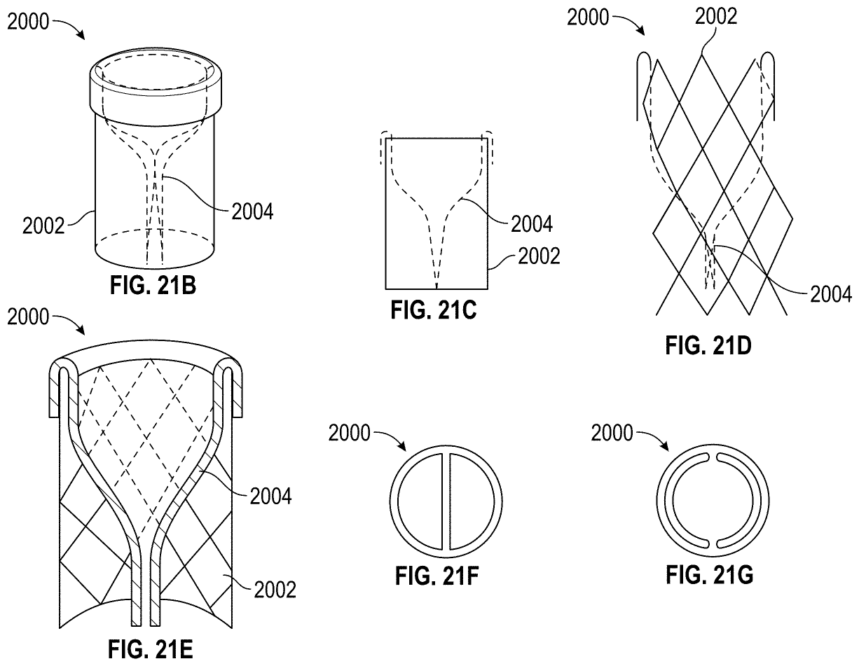

FIG. 11 shows various components that may be incorporated into system 600 described above that are not visible in FIGS. 13A-13B and that are configured to operate LA balloon 102 and PA balloon 104 as described herein. Further details regarding components of the system 100 are also described in U.S. Provisional Application No. 62/801,917, filed Feb. 6, 2019, including but not limited to FIG. 21 and paragraph [0057], the entirety of which is hereby incorporated by reference. In the example of FIG. 11, system 600 may include control circuitry (not shown), a power source (not shown), a pressure chamber or reservoir 1900, a vacuum chamber or reservoir 1902, and a pump 1907. As shown, solenoids 1908, 1909 may be disposed on tubing that fluidly couples pressure chamber 1900 and vacuum chamber 1902 to fluid lines (e.g., implementations of fluid lines 110 and 112 of FIGS. 13A-13B) can be controlled by control circuitry at microprocessor 1927 to control the inflation and deflations of balloons 102 and 104. In one embodiment, ECG sensors 1903 are connected to the patient 1901 and the patient's ECG signal is sent to the data acquisition unit 1905 (Power Lab), which is programmed to look for a set threshold value that correlates to the R-wave in the ECG signal. Once the threshold is detected, the data acquisition unit 1905 sends a pulse (square wave) to a microcontroller 1927. The software 1915 monitors the microcontroller 1927 for the pulses sent by the data acquisition unit 1905 and uses that information to continuously calculate the interval between R-waves (the R-R interval) of the ECG signal. The PA and LA balloon inflation is timed using the calculated R-R intervals and the parameters 1919, 1929 (including length of inflation time, offset/delay time after detection of ECG feature 1917, and fill volume), which are adjusted with the user input/controller 1921. Based on the R-R interval timing and the user input 1921, the software then communicates with the microcontroller 1927 to actuate the solenoids 1908, 1909, opening the balloon lumen(s) to either the pressure chamber 1900 for inflation, or the vacuum chamber 1902 for deflation.

Although system 600 is depicted as an external fixed system (e.g., for bedside support), the components of FIG. 11 and the other figures described above can also be arranged for ambulatory use, or for implantation in the patient (e.g., the drive system for balloons 102 and 104 can be in an external console, a wearable external portable unit, or could be fully implantable). System 600 can provided for temporary, short-term, mid-term, long-term, or permanent use. In temporary cases, LA and PA positioning structures 106 and 108 are arranged to be removed from the patient atraumatically.

If desired, balloons 102 and/or 104 can be provided with a pressure sensor/monitor 1923, 1931 that collect pressure data within the corresponding cavity. Pressure data from these pressure sensors can be used to drive or trigger the balloon inflation and/or deflation and/or can be collected to provide information to the patient, physician, or others in real-time via an output display 1925 or when uploaded separately. In some embodiments, sensors 1923, 1931 may also be used to monitor pressure inside the balloons for various purposes.

Although various examples are discussed herein in which LA pressurizing element 102 and PA pressurizing element 104 are implemented as balloons, it should be appreciated that dual-sided system 600 can be implemented with other pressurizing elements such as active pumps, axial flow pumps, turbines, or other mechanisms for displacing volume and fluids. More generally, each of elements 102 and 104 can be implemented as any suitable combination of pressure-control, fluid-displacement, and/or volume-displacement mechanisms that are biocompatible and implantable for positioning in fluid communication with one or more portions of the left or right side of a patient's heart. For example, LA pressurizing element 102, when operated, may cause a volume displacement in the LA, and PA pressurizing element 104, when operated, may cause a volume displacement in the Pulmonary Artery. As would be understood by one of ordinary skill in the art, the left side of the heart includes the LA and the Left Ventricle, and receives oxygen-rich blood from the lungs and pumps the oxygen-rich blood to the body. As would be understood by one of ordinary skill in the art, the right side of the heart includes the right atrium and the right ventricle, and receives blood from the body and pumps the blood to the lungs for oxygenation.

Similar to the single balloon system described above, FIG. 12 conceptually illustrates an electronic system with which one or more aspects of the subject technology may be implemented. Electronic system, for example, may be, or may be a part of, control circuitry 1913 for a dual-sided cardio-pulmonary support system implemented in standalone device, a portable electronic device such as a laptop computer, a tablet computer, a phone, a wearable device, or a personal digital assistant (PDA), or generally any electronic device that can be communicatively coupled to pressurizing devices implanted in a patient's heart and or pulmonary vasculature. Such an electronic system includes various types of computer readable media and interfaces for various other types of computer readable media. Electronic system includes bus 1008, processing unit(s) 1012, system memory 1004, read-only memory (ROM) 1010, permanent storage device 1002, input device interface 1014, output device interface 1006, and network interface 1016, or subsets and variations thereof.

Bus 1008 collectively represents all system, peripheral, and chipset buses that communicatively connect the numerous internal devices of electronic system. In one or more embodiments, bus 1008 communicatively connects processing unit(s) 1012 with ROM 1010, system memory 1004, and permanent storage device 1002. From these various memory units, processing unit(s) 1012 retrieves instructions to execute and data to process in order to execute the processes of the subject disclosure. The processing unit(s) can be a single processor or a multi-core processor in different embodiments.

ROM 1010 stores static data and instructions that are needed by processing unit(s) 1012 and other modules of the electronic system. Permanent storage device 1002, on the other hand, is a read-and-write memory device. This device is a non-volatile memory unit that stores instructions and data even when electronic system is off. One or more embodiments of the subject disclosure use a mass-storage device (such as a magnetic or optical disk and its corresponding disk drive) as permanent storage device 1002).

Other embodiments use a removable storage device (such as a floppy disk, flash drive, and its corresponding disk drive) as permanent storage device 1002. Like permanent storage device 1002, system memory 1004 is a read-and-write memory device. However, unlike storage device 1002, system memory 1004 is a volatile read-and-write memory, such as random access memory. System memory 1004 stores any of the instructions and data that processing unit(s) 1012 needs at runtime. In one or more embodiments, the processes of the subject disclosure are stored in system memory 1004, permanent storage device 1002, and/or ROM 1010. From these various memory units, processing unit(s) 1012 retrieves instructions to execute and data to process in order to execute the processes of one or more embodiments.

Bus 1008 also connects to input and output device interfaces 1014 and 1006. Input device interface 1014 enables a user to communicate information and select commands to the electronic system and/or a sensor to communicate sensor data to processor 1012. Input devices used with input device interface 1014 include, for example, alphanumeric keyboards, pointing devices (also called “cursor control devices”), cameras or other imaging sensors, electro-cardio sensors, pressure sensors, or generally any device that can receive input. Output device interface 1006 enables, for example, the display of images generated by electronic system. Output devices used with output device interface 1006 include, for example, printers and display devices, such as a liquid crystal display (LCD), a light emitting diode (LED) display, an organic light emitting diode (OLED) display, a flexible display, a flat panel display, a solid state display, a projector, or any other device for outputting information. One or more embodiments may include devices that function as both input and output devices, such as a touch screen. In these embodiments, feedback provided to the user can be any form of sensory feedback, such as visual feedback, auditory feedback, or tactile feedback; and input from the user can be received in any form, including acoustic, speech, or tactile input. Output device interface 1006 may also be used to output control commands for operating pressurizing components (e.g., to control pressurizing elements 102 and 104) as described herein.

Finally, as shown in FIG. 12, bus 1008 also couples electronic system to a network (not shown) through network interface 1016. In this manner, the computer can be a part of a network of computers (such as a local area network (“LAN”), a wide area network (“WAN”), or an Intranet, or a network of networks, such as the Internet. Any or all components of electronic system can be used in conjunction with the subject disclosure.

### Pulmonary Vein Shield(s)

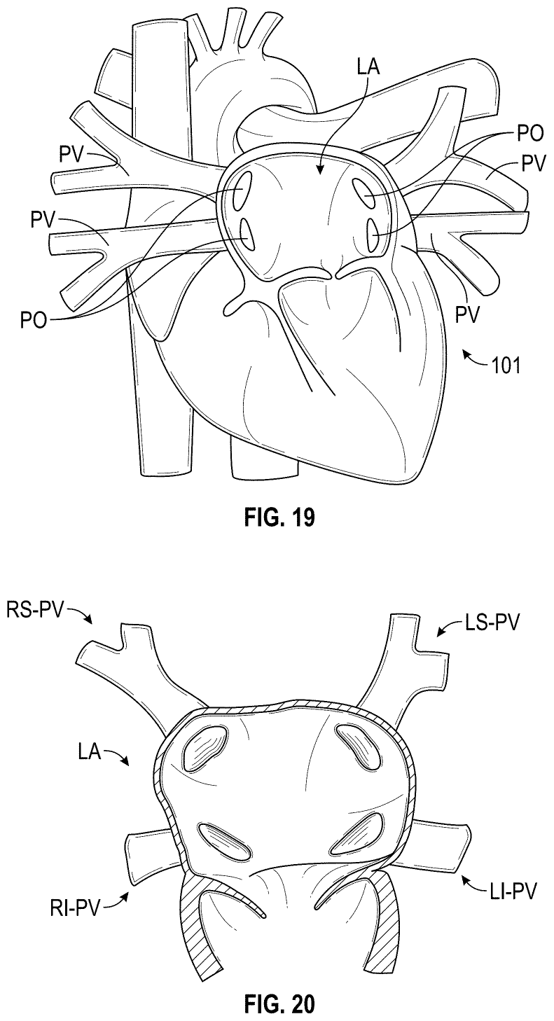

Pulmonary shields or blood-regulating valves will now be described, which may be utilized independently or with any of the systems described above. When the pulmonary shield is used with a cardiac support system described above, the pulmonary shield can prevent a backflow of blood into the pulmonary veins PV and a spike in PCWP if the assisted atrial systole results in a higher a-wave LA pressure. A shield or blood-regulating valve may be positioned in a LA of a heart 101, as shown in FIG. 19. As further described below, the shield may engage with, cover, and/or block flow into the ostia PO of any one or more of the different pulmonary veins PV, such as right superior pulmonary vein RS-PV, left superior pulmonary vein LS-PV, right inferior pulmonary vein RI-PV, and the left inferior pulmonary vein LI-PV, shown in FIG. 20.

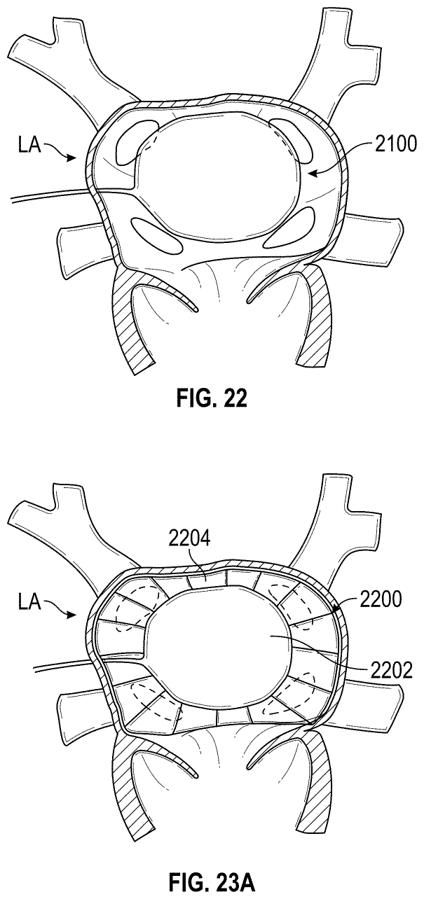

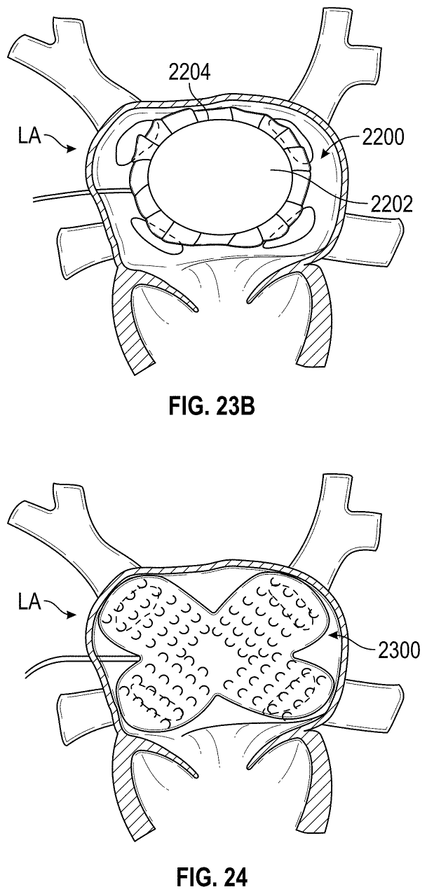

The shield or blood-regulating valve may take a variety of forms including but not limited to: individual one-way valve assemblies 2000 that can be positioned inside each of the pulmonary ostia PO (shown in FIGS. 21A-21G), a two or three dimensional non-porous surface 2100 comprising a single layer that can obstruct or divert flow (shown in FIG. 22), a two or three dimensional surface 2200 with a non-porous layer 2202 in the center and blood regulating flaps 2204, or similar construction, around the perimeter and/or in the center of the non-porous layer 2202, that can regulate flow (shown in FIG. 23A with the flaps 2204 closed and in FIG. 23B with the flaps open 2204), a two or three dimensional surface 2300 with selective porosity that can allow flow from the pulmonary veins PVs to the LA but restrict or reduce flow from the LA to the pulmonary veins PVs (e.g., a quatrefoil shape shown in FIG. 24), or any combination of the aforementioned alternatives. In these or other embodiments, the shield or blood-regulating valve may be configured to allow for blood to flow from the lungs into the LA and to partially or completely restrict blood from flowing from the LA into the lungs. Additionally, these or other embodiments of the shield or blood regulating valve can be configured in such a way that when the shield or blood regulating valve is positioned off of the surface of the PV ostia PO, an increase in the LA pressure can drive the shield or blood regulating valve to at least partially close off the PV ostia PO, and when the LA pressure decreases, the shield or blood regulating valve can move such that the PV ostia PO opens back up. The shield or blood-regulating valve may be collapsible for delivery to the desired cardiac location and then expandable into its desired shape and configuration. In some embodiments, such as shown in FIGS. 21A-24 and 25A-28D and other embodiments of this specification, the shield or blood-regulating valve may be positioned at a distal end of a delivery device such as a wire, catheter or sheath for intravascular delivery to the desired location (e.g., the LA).

FIGS. 21A-21G illustrate individual one-way valve assemblies 2000 that can be positioned inside each of the pulmonary ostia PO, as shown in FIG. 21A. The one way valve assembly 2000 can comprise an expandable frame 2002 and a one way valve 2004 (e.g., a duckbill valve or any suitable one-way valve). The expandable frame 2002 can be configured to expand within the pulmonary veins PV such that the one way valve assembly 2000 is secured within the pulmonary veins PV. In some configurations, the expandable frame 2002 can comprise a shape-set wire, laser cut sheet or tube, molded material or any other material for compression into a catheter and re-expansion when delivered to the pulmonary vein PV. In some configurations, the one way valve 2004 can be positioned radially inward from the expandable frame 2002. In some configurations, the one way valve 2004 can be configured to allow blood to flow from the pulmonary veins PV to the LA and prevent blood from flowing from the LA to the pulmonary veins PV.

As shown in FIGS. 21B-21E, the shape of the one-way valve 2004 can be configured to allow the valve 2004 to open when a pressure differential drives blood flow from the pulmonary veins PV to the LA. Additionally, a pressure differential that drives blood flow from the LA to the pulmonary veins PV can cause the valve 2004 to close. FIGS. 21F and 21G show a bottom view of the one-way valve assembly 2000 in a closed configuration and in an open configuration, respectively. In some configurations, the one-way valve 2004 can be made of a membrane, fabric, or other thin, non-porous material that can be affixed to the expandable frame 2002. As shown in FIGS. 21B-21E, a first end of the one-way valve 2004 (e.g., that the end closest to the pulmonary veins PV when implanted) can be folded radially outward over an outer surface of the expandable frame 2002 to secure the one-way valve 2004 to the expandable frame 2002. In some embodiments, the one-way valve 2004 can comprise a flexible material configured to at least partially conform to the expandable frame 2002.

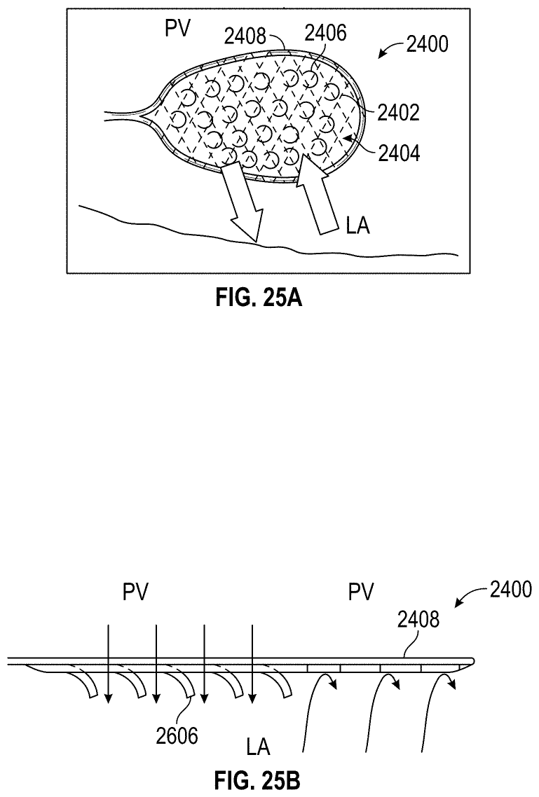

FIGS. 25A-25B show an embodiment of a shield 2400 comprising multiple layers 2402, 2404 that can provide for a porous regulating surface. The multiple layers 2402, 2404 can comprise a back PV facing layer or backing layer 2402 that can be at least partially porous and a front LA facing layer 2404 that can be non-porous. The non-porous layer 2404 may comprise a membrane that can include flaps 2406. The flaps 2406 can be cut into the membrane or by any suitable method. The flaps 2406 can be configured to open away from the porous backing layer 2402 towards the LA to allow flow from the pulmonary veins PV to the LA when there is a suitable pressure differential. When LA pressure increases and exceeds PV pressure, the flaps 2406 can close back against the backing layer 2404 thereby restricting flow into the pulmonary veins PV and avoiding a spike in pressure and reducing the average PCWP. Various materials could be used for each layer 2402, 2404 including woven or knit fabric, polymer membranes, metal mesh, and/or others. The shield 2400 may further comprise a structural support 2408 such as a wire frame that can be configured to support the multiple layers 2402, 2404. The shield 2400 may be collapsible for intravascular delivery, such as within a delivery catheter or sheath, and be expandable within the LA to cover one or more ostia PO of the pulmonary veins PV.

In some embodiments, the surfaces of the shield 2400 facing the PV ostia PO and the LA may be two dimensional (e.g., substantially planar) or may have a three dimensional shape when expanded. For example, the surface of the shield 2400 may be shaped so as to generally conform to at least a portion of the interior surface of the LA for a better seal. In some embodiments, the surface of the shield 2400 can be shaped to cover all four PV ostia PO, while in other embodiments, the surface of the shield 2400 may be shaped to cover only one, two, or three PV ostia PO. In some embodiments, the shield 2400 can be configured to avoid restriction of flow through the mitral valve. The outer perimeter of the shield 2400 may have a variety of different shapes to cover one or more of the PV ostia PO, such as, but not limited to, circular, oval, clover, butterfly, and quatrefoil. In some embodiments, the surface of the shield 2400 can include individual concave regions such that the surface of the shield 2400 can extend deeper into the PV ostia PO from the LA region for better seating within the ostia PO and to reduce the risk of a diaphragm effect that could cause undesirable pressure transference to the pulmonary veins PVs even when the one-way valve is closed.

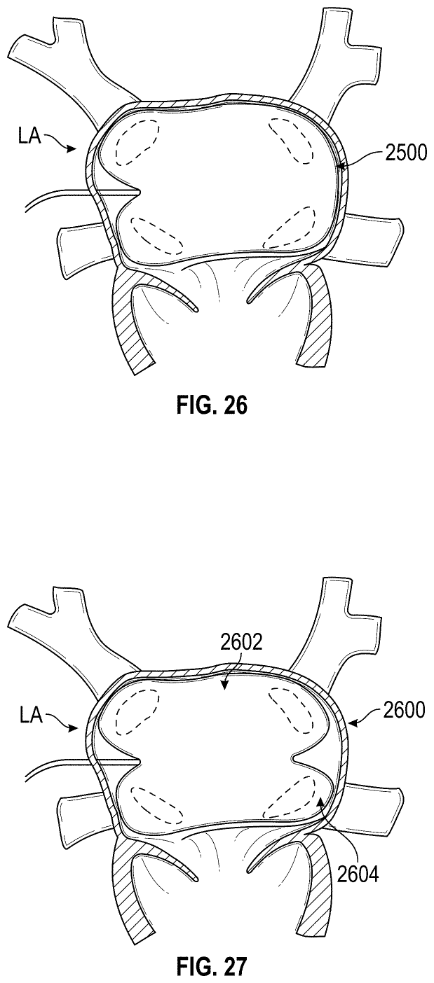

FIGS. 26-28D show multiple embodiments of a surface-type shield that can include a perimeter formed from a shape-set wire, laser cut sheet, molded material or any other structural support element suitable for compression and re-expansion into a catheter. This system of these or any other embodiments described herein may be introduced from a catheter inserted from the venous system (for example, trans-femoral or trans-jugular) and positioned trans-septally between the right atrium RA and LA for deployment inside the LA. A variety of shapes could be utilized such that a single shield or multiple shields can cover all four pulmonary vein ostia PO or a subset thereof. As shown in FIG. 26, a shield 2500 may comprise a single lobe that covers all four pulmonary vein ostia PO. As shown in FIG. 27, a shield 2600 may comprise two lobes 2602, 2604. The two lobes 2602, 2604 can include a first lobe 2602 that can be configured to cover the right and left superior pulmonary vein ostia and a second lobe 2604 that can be configured to cover the right and left inferior pulmonary vein ostia.

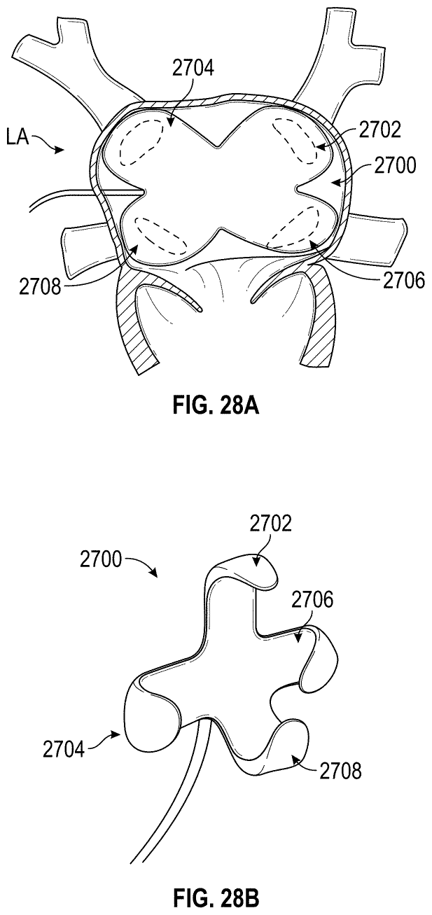

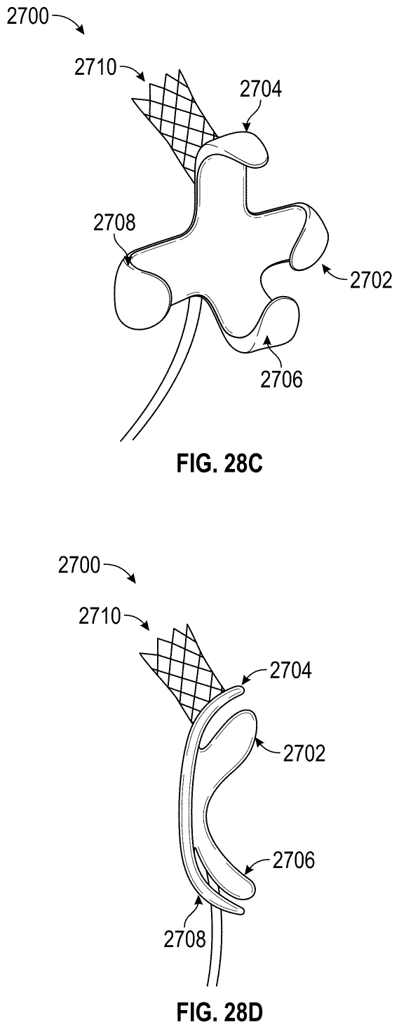

As shown in FIGS. 28A-28D, a shield 2700 may comprise four lobes 2702, 2704, 2706, 2708. The four lobes 2702, 2704, 2706, 2708 can comprise a first lobe 2702 configured to cover the right superior pulmonary vein ostia, a second lobe 2704 configured to cover the left superior pulmonary vein ostia, a third lobe 2706 configured to cover the right inferior pulmonary vein ostia, and a fourth lobe 2708 configured to cover the left inferior pulmonary vein ostia. In some embodiments, the surface-type shield 2700 can further comprise one or more one-way valves on, attached to, or otherwise coupled to at least one of the lobes 2702, 2704, 2706, 2708 of the shield 2700, such as the one-way valve assemblies 2000 described in relation to FIGS. 21B-21G. For example, the illustrated configuration shows a one way valve assembly 2710, which can be the same or similar to the one way valve assembly 2000, attached to the second lobe 2704 of the shield 2700. The benefits of attaching the one or more one-way valve assemblies 2710 to one or more lobes 2702, 2704, 2706, 2708 of a surface-type shield 2700 include: facilitating delivery of the one-way valve assemblies 2710 into the pulmonary veins PV, reducing the likelihood of migration of the one-way valve assemblies 2710 from the pulmonary veins PV into the LA, and allowing for removal of the one-way valve assemblies 2710 from the pulmonary veins PV.

The shields 2500, 2600, 2700 shown in FIGS. 26-28D may be formed from a single wire, multiple wires, or any other structural support element described above such that the shape and/or size of each lobe can be modified independently either pre-procedurally or in situ to best conform to the patient's anatomy. The shields 2500, 2600, 2700 may comprise multiple layers such as described with respect to FIGS. 25A-25B or may comprise other structures for regulating blood flow as described elsewhere in this specification.

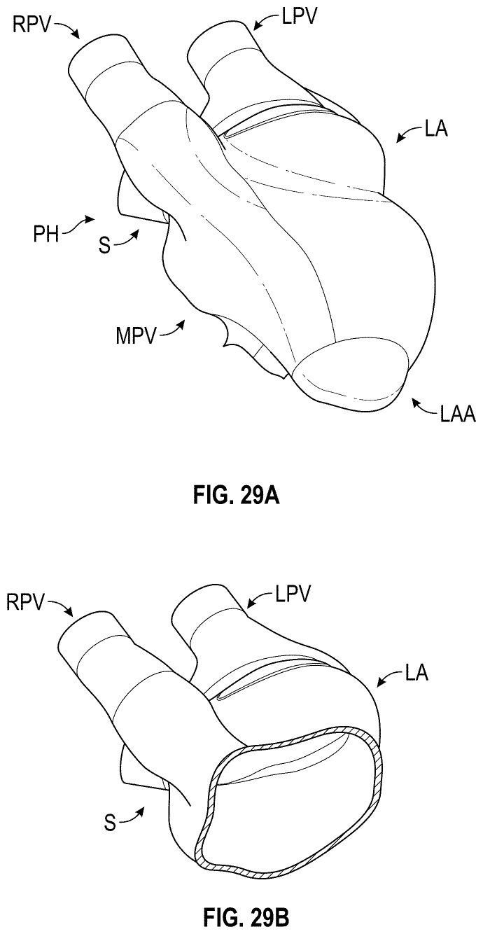

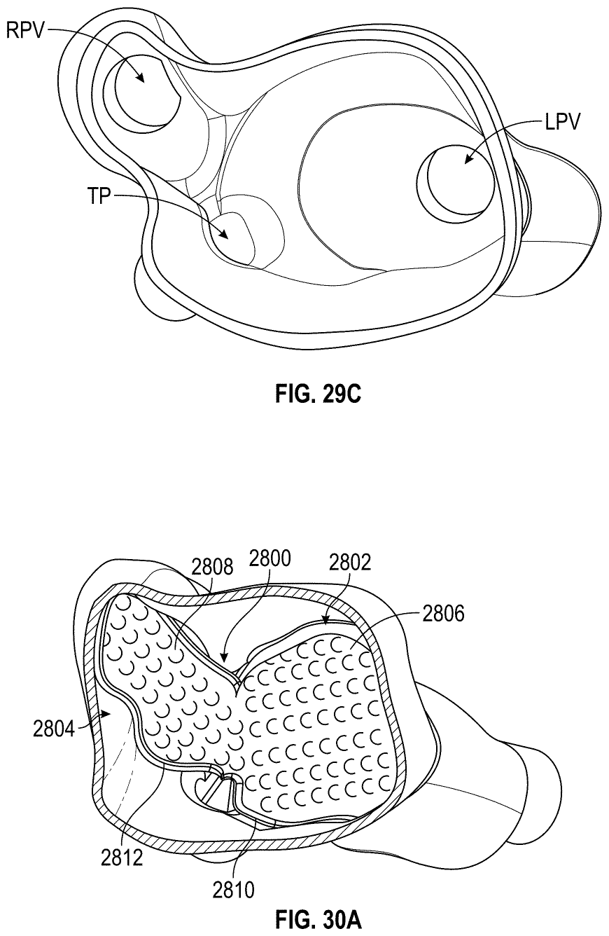

FIGS. 29A-29C show a model of a porcine heart PH with various ports labeled. For example, FIG. 29A shows the right pulmonary vein RPV, the left pulmonary vein LPV, the LA, the septum S, the mitral valve plane MVP, and the left atrial appendage LAA of the porcine heart PH. FIG. 29B illustrates the porcine heart PH with certain portions removed (e.g., all portions at and below the mitral valve of the porcine heart PH including the left atrial appendage LAA). FIG. 29C illustrates a bottom view of the porcine heart PH shown in FIG. 29B with a transseptal puncture TP created in the porcine heart PH. While the human heart 101 has four pulmonary veins PV, the porcine heart PH has two pulmonary veins PV. Nonetheless the porcine heart PH can be a suitable model for evaluating the utility of potential designs. Designs that function well in the porcine heart PH can then be modified further to accommodate the human heart 101. Moreover, embodiments of the shields described herein may be suitable for use in animal testing.

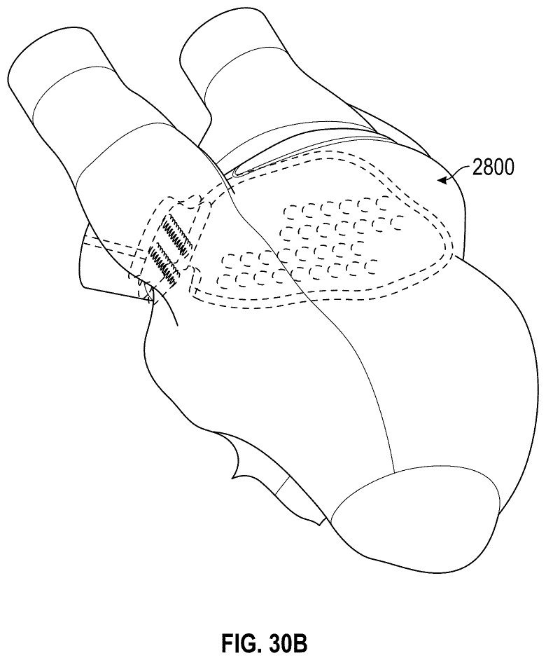

FIGS. 30A-30B show an embodiment of a surface shield 2800 configuration with two wire-formed lobes 2802, 2804 comprising a left lobe 2802 and a right lobe 2804. Each of the lobes 2802, 2804 can comprise a membrane 2806, 2808 configured to cover the ostia PO of the pulmonary veins PV and a wire-form 2810, 2812 surrounding each membrane 2806, 2808. The membranes 2806, 2808 can be configured to regulate flow through the ostia PO of the pulmonary veins PV. The two wire-formed lobes 2802, 2804 can extend from the distal end of a delivery device, such as a delivery shaft that may be delivered through a trans septal opening TP between the right and left atria RA, LA. Each wire-formed lobe 2802, 2804 may be shaped to conform to the particular ostia PO of the pulmonary vein PV in which the lobe 2802, 2804 is being situated, and thus may have different shapes. As described above, the different lobes 2802, 2804 can be formed independently pre-procedurally or in situ to best conform to the surrounding anatomy for an optimal seal.

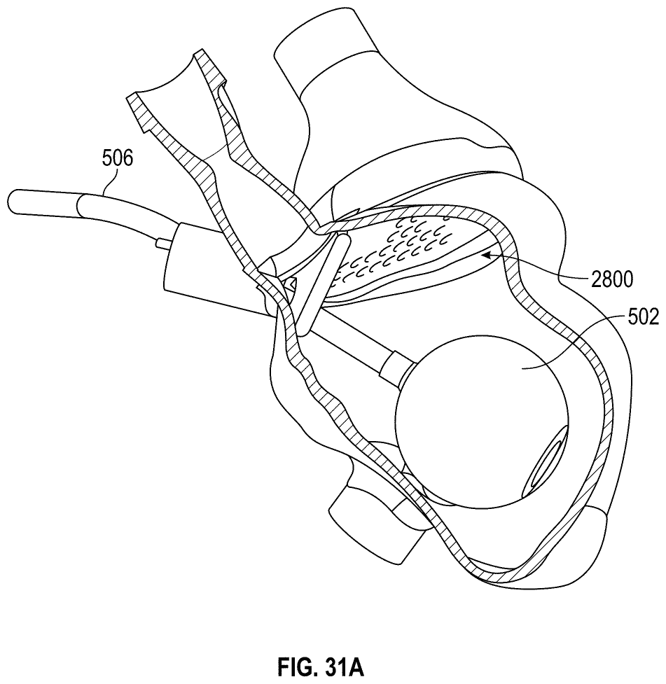

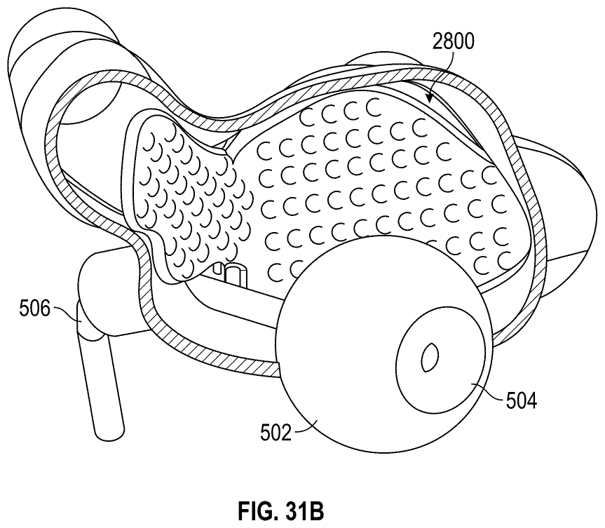

Pulmonary vein shields as described above can be utilized in conjunction with intra-cardiac support systems used to treat heart failure, such as the systems described in relation to FIGS. 2-18. FIGS. 31A-31B show the shield configuration shown in FIG. 30 in conjunction with the LA balloon 502 shown in FIGS. 10A-10B. The inflation of the LA counterpulsation balloon 502 during atrial systole may supplement the native atrial contraction and lead to additional forward flow and filling of the ventricle during end diastole before the mitral valve closes. The presence of a pulmonary valve shield 2800 in this case, may prevent a spike in PCWP in the event that the assisted atrial systole results in a higher a-wave (peak) LA pressure. In some embodiments, the balloon 502 and the pulmonary vein shield 2800 can both be configured such that after inflation of the balloon 502 and expansion of the shield 2800, the balloon 502 and shield 2800 do not interfere with one another.

In some configurations, a single transseptal sheath can contain both the pulmonary valve shield 2800 and the LA balloon 502. In some configurations, the shield 2800 can be loaded distally to the LA counterpulsation balloon 502 inside the same deployment sheath. In some configurations, the LA counterpulsation balloon 502 can be loaded in the same catheter on top of the pulmonary valve shield 2800 or the LA counterpulsation balloon 502 can be loaded in a different catheter than the pulmonary valve shield 2800 and exchanged through the same transseptal sheath or over the same transseptal wire. In some configurations, the LA counterpulsation balloon 502 and the pulmonary valve shield 2800 can be delivered through separate transseptal sheaths.

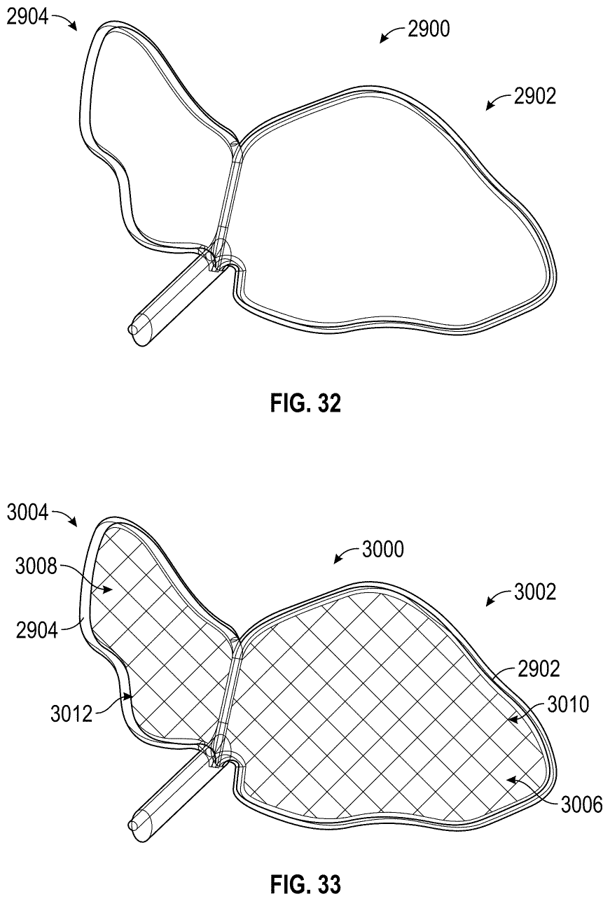

FIG. 32 shows the shape of one possible wire-formed shield 2900 in an expanded configuration after exiting a delivery sheath. The shield 2900 can include a first larger lobe 2902 and a second smaller lobe 2904. In some configurations, the lobes 2902, 2904 may be the same size and there may be fewer or a greater number of lobes (e.g., 1, 3, or 4). Each lobe 2902, 2904 may have a two or three dimensional perimeter shape that can include multiple bends or curves to conform to native anatomy. In some configurations, one or more lobes can have different shapes to accommodate the native anatomy, while in other configurations, the plurality of lobes can have the same shape to facilitate manufacturing and ease of deployment.

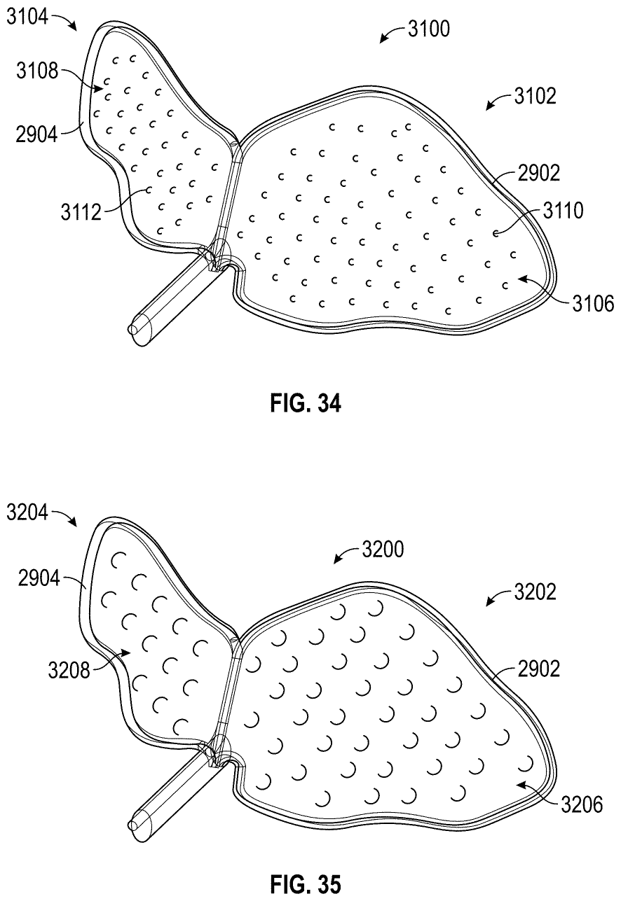

FIGS. 33-34 show different embodiments of a shield 3000, 3100 with porous backing layers. FIG. 33 shows a possible configuration of the shield 3000 that can comprise a porous backing layer 3006, 3008 and a mesh layer 3010, 3012 supported by the wire frame 2902, 2904 of FIG. 32. FIG. 34 shows an embodiment of the shield 3100 that can comprise a membrane 3106, 3108 with perforations 3110, 3112 being supported by the wire frame 2902, 2904. The porous backing layers can be designed to account for the tradeoff between minimization of forward flow (i.e., from pulmonary vein PV to LA) gradient versus the valve-functionality of the shield 3000, 3100 (e.g., the non-porous layer 2404 described in relation to FIGS. 25A-25B including flaps 2406 that open and close against the backing layer 2402) and the ability of the valve (e.g., the flaps 2406) to seal against the backing layer. For example, the wire mesh 3010, 3012 shown in FIG. 33 may provide for greater forward flow than the membrane 3106, 3108 of FIG. 34, but it may be more difficult to obtain a complete seal of the valve against the wire mesh 3010, 3012. With the porous membrane 3106, 3108 shown in FIG. 34, it may be easier for the valve to form a seal due to the greater surface area, but forward flow may be minimized. The level or porosity of the backing layer in these embodiments can be modified in order to obtain a desired balance between minimizing forward flow (i.e., from the pulmonary vein PV to LA) gradient and maximizing backflow sealing.

FIGS. 34-37 show a variety of possible configurations 3200, 3300, 3400 for a non-porous cut membrane 3206, 3208, 3306, 3308, 3406, 3408 on the wire-formed shield 2900 (shown FIG. 32) to regulate blood flow in front (i.e., on the LA side) of the porous backing material. Each of the shields 3200, 3300, 3400 can include a first lobe 3202, 3302, 3402 and a second lobe 3204, 3304, 3404. As shown in these figures, the direction and orientation of the cuts and hinge points can vary in order to optimize flow in one direction with a minimal gradient and minimize flow in the opposite direction.

In some configurations, the flaps in the shields 3200, 3300, 3400 and the other shields described elsewhere in the specification can be configured to open over a hole so that the flaps can close against a surface with more of an overlapping contact to obtain better sealing. In some configurations, the flaps can be created with an angled cut through the wall of the membrane 3206, 3208, 3306, 3308, 3406, 3408 that could allow the flaps to close against the wall surface 3206, 3208, 3306, 3308, 3406, 3408 of the membrane material and get more overlap for better sealing. In some configurations, the flaps can be created in the membrane 3206, 3208, 3306, 3308, 3406, 3408 by laser cutting or any other suitable method. In some configurations, the shield 3200, 3300, 3400 can comprise only a single porous layer with flaps that are configured to only open in one direction, for example, using one of the techniques previously described (e.g., angled cuts or hinges).

In some configurations, shields 3200, 3300, 3400 and the other shields described elsewhere in the specification can comprise a non-circular shape that can be controllably released such that a necked-down portion in the middle of the shield can expand in situ and appose the surrounding anatomy. In this configuration, the shield 3200, 3300, 3400 can include a two lobe shape made from a single wire that can be held in a necked down position at the center (i.e., a peanut shell shape), which can then be expanded so that the peanut shell shape can expand into a circular or oval-like shape. In some embodiments, the shield can be flat, concave and/or have one end out of plane with respect to the other.

In some configurations, the shield 3200, 3300, 3400 and the other shields described elsewhere in the specification can include multiple overlapping wires that can be used to form the perimeter of the shield 3200, 3300, 3400 to facilitate collapsing the system and loading it into a catheter while ensuring that there are no gaps in the perimeter and that apposition around a given circumferential cross-section of the cavity is maintained. In some configurations, the shield 3200, 3300, 3400 can comprise two overlapping wires that can control the lobe shapes. By overlapping the two wires, the lobe shapes can be independently controlled while avoiding having a divot at the top that would result in a heart shape, which allows the perimeter of the shield 3200, 3300, 3400 to have more of a continuous contact along the top if desired.

In some configurations, the shield 3200, 3300, 3400 and the other shields described elsewhere in the specification can comprise a non-circular shape that can be controllably released so that a necked-down portion in the middle of the shape expands in situ and apposes the surrounding anatomy. In some configurations, the shield 3200, 3300, 3400 can comprise a preformed support tube from which the wires could extend and form the lobes 3202, 3204, 3302, 3304, 3402, 3404. The preformed support tube can include one or more offset holes that can be preferentially oriented in order to maintain a desired angle between the two side lobes. The offset holes in the preformed support tube may offer more control via the tube and more torque control to the system.

In some configurations, the shield 3200, 3300, 3400 and the other shields described elsewhere in the specification can have different surface configurations. In some configurations, the sides of the surface (e.g., two adjacent lobes 3202, 3204, 3302, 3304, 3402, 3404) can be configured to roll up like a scroll in order to facilitate loading and deployment of the shield 3200, 3300, 3400. In some configurations, the shield 3200, 3300, 3400 can be loaded and/or deployed from the catheter by rolling it up (e.g., like a single tube), closing it (e.g., like a paper fan), pulling the lobes 3202, 3204, 3302, 3304, 3402, 3404 down on the sides from a central axis reference, and/or pushing the lobes 3202, 3204, 3302, 3304, 3402, 3404 up on the sides from a central axis.

As shown in FIG. 37, reinforcement elements 3410, 3412 may be added to the surfaces 3406, 3408 in order to provide additional support during loading and release in high strain areas along the surface 3406, 3408. The reinforcing element 3410, 3412 can be made from wire, suture, polymer or any other structural support material or by maintaining uncut regions or heavier thickness regions of the front and/or back surfaces in order to maintain greater structural support along the direction of tension.

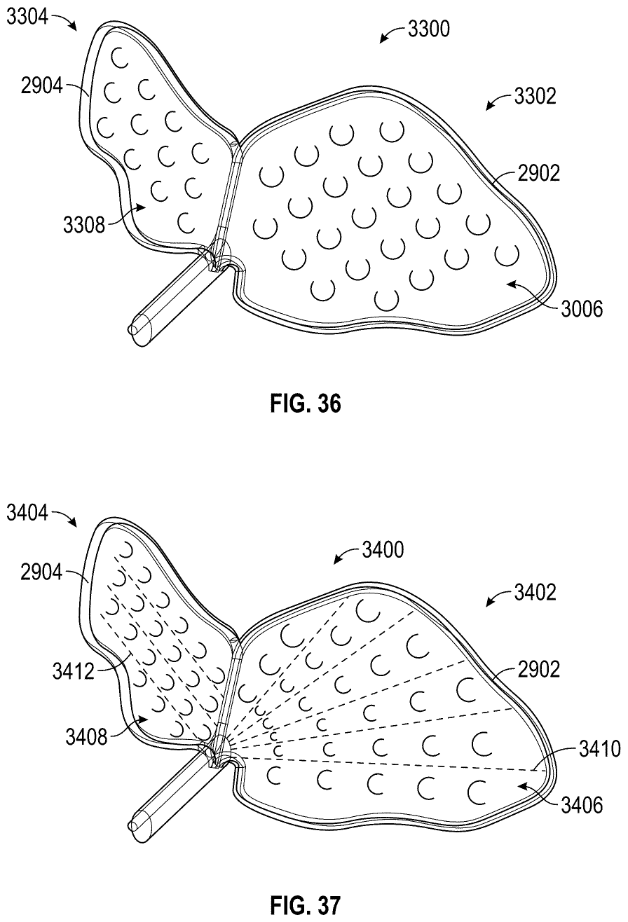

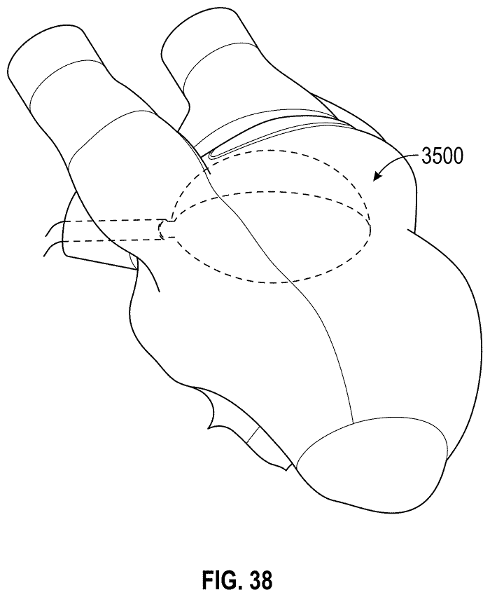

FIG. 38 shows a possible three-dimensional shape for support scaffolding 3500 for a pulmonary vein shield to bias the surface of the shield towards the ostium PO of a pulmonary vein PV. The support scaffolding 3500 can be ellipsoid in shape or it can be at least partially bowl-shaped such that it generally conforms to the interior surface of the LA for a better seal. Part of the surface of the support scaffolding 3500, such as the bowl-shaped portion, may be covered by the porous and/or non-porous layers described above. Such an approach could minimize additional bowing of the surface towards the pulmonary vein PV when the blood regulating surface is closed. In other words, this approach can prevent the closed surface of the shield from functioning as a diaphragm, or a trampoline, where displacing the closed surface towards the pulmonary vein PV could result in a compression of volume in the pulmonary vein PV and elevate the pressure. The bowl shape of the shield 3500 can also facilitate placement of the LA pressurizing element inside the LA without contacting the shield 3500.

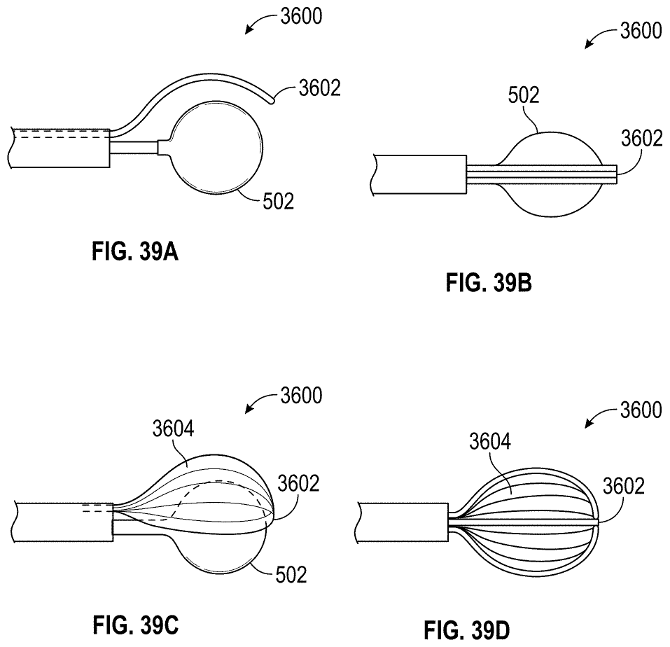

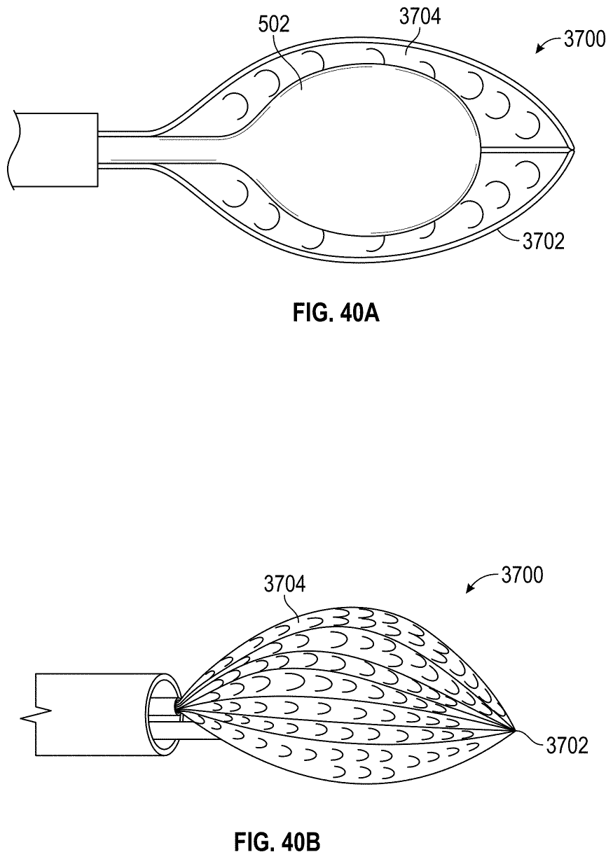

FIGS. 39A-40B show three dimensional embodiments of a pulmonary vein shield 3600, 3700 comprising a frame 3602, 3702 configured to encompass at least a portion of the LA. The blood regulating surfaces 3604, 3704 of the shield 3600, 3700 can extend over the frame or cage 3602, 3702. In some configurations, a LA balloon 502 can be located inside the frame/cage 3602, 3702. In some configurations, the LA balloon 502 (e.g., the distal end of the LA balloon 502) can be secured to the cage 3602, 3702 (e.g., the right side of the cage 3602, 3702) for stability. The LA balloon 502 can have a guidewire running through it to aid in securement. In some configurations, the LA balloon 502 can be inside the frame/cage 3602, 3702 as the frame/cage 3602, 3702 is deployed within the LA or the LA balloon 502 can be advanced into the frame/cage 3602, 3702 after the frame/cage 3602, 3702 is deployed in position.

In some configurations, the frame/cage 3602, 3702 can be deployed in a compressed configuration with the LA balloon 502. For example, FIGS. 39A-39D illustrate a two-step method of deploying the shield 3600 with the LA balloon 502. As shown in FIGS. 39A-39B, the compressed configuration of the shield 3600 can be delivered with the LA balloon 502. Once the shield 3600 and the LA balloon 502 are within the LA, the shield 3600 can be expanded into an expanded configuration to at least partially cover the LA balloon 502, as shown in FIGS. 39C-39D. In some configurations, the shield 3600 can include a frame 3602 made of a plurality of longitudinal ribs that expand from a compressed configuration (e.g., from within a delivery catheter), as shown in FIGS. 39A and 39B, to an expanded configuration, as shown in FIGS. 39C and 39D. The expanded frame 3602 can create a concave surface oriented towards the LA and a convex surface oriented towards the pulmonary veins PV. In some configurations, the frame 3602 can resemble a semispherical shape, although in other configurations, the shape may be more or less than half a sphere, may be non-spherical (e.g., ellipsoid), may be non-uniform, and/or may conform to the anatomy of the LA.

In some configurations, the shield 3600 can further include a second layer on the concave side of the frame 3602 that can be configured to allow blood to flow from the pulmonary veins PV to the LA, and to prevent blood flow from the LA to the pulmonary veins PV. In some embodiments the second layer includes one-way valve flaps, such as any of the embodiments of flaps described herein. The number and spacing of the longitudinal ribs of the frame 3602 as well as the size and spacing of the flaps can be adjusted to allow the flaps of the second layer to close against at least one rib of the frame, such that the flaps close against the ribs and the second layer becomes non-porous when blood flows from the direction of the LA to the pulmonary veins PV.

The three dimensional pulmonary vein shield 3600, 3700 can comprise different configurations. For example, the three dimensional pulmonary shield 3600, 3700 can include a full cage surrounding the LA balloon 502 or a funnel shaped cage surrounding the LA balloon 502. In some configurations, the three-dimensional shield surface 3604, 3704 can be configured to be positioned behind the LA balloon 502 (i.e., toward the pulmonary veins PV) such that the LA balloon 502 can sit in front of the surface 3604, 3704 in the LA and avoid interacting with the shield 3600, 3700 when the balloon 502 expands. In some configurations, the shield's surface 3604, 3704 can be concave towards the balloon 502 so that the shield's surface 3604, 3704 does not interfere with the inflation of the balloon 502.

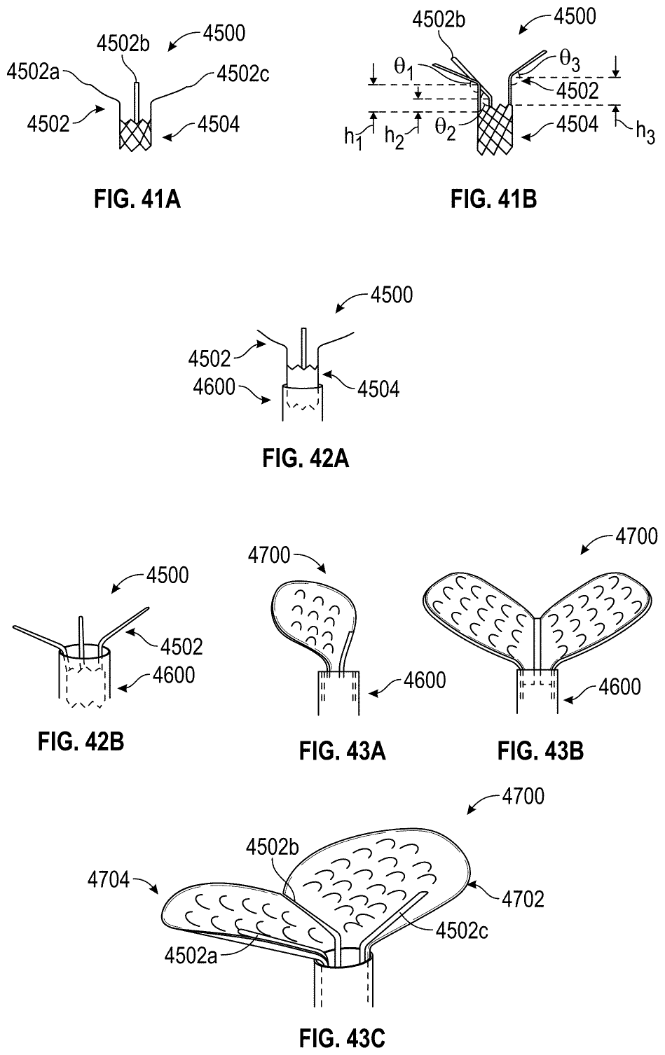

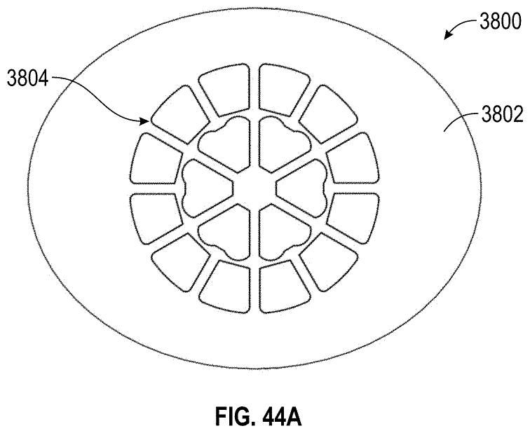

As shown in FIGS. 41A-41B, a shield 4700 (shown in FIGS. 43A-43C) can include a support 4500. In some configurations, a plurality of support features or posts 4502 can be incorporated into a body 4504 of the support 4500. In some configurations, the body 4504 can comprise an expandable stent (e.g., a wire form or a laser cut member) or a solid tube with a circumferential shape. The body 4504 can be attached to the surface of the shield 4700 in order to maintain a position inside an introducer sheath 4600 (e.g., FIGS. 43A-43C), either for direct concentricity or to maintain a desired radial or circumferential offset. In some configurations, the plurality of support posts 4502 can include a first support post 4502a, a second support post 4502b, and a third support post 4502c. Although the illustrated configuration shows three support posts 4502a, 4502b, 4502c, the plurality of support posts 4502 can comprise more or less than three support posts (e.g., two, four, five, six, etc.) or there can be a single support post.

As shown in FIG. 41B, each of the plurality of support posts 4502a, 4502b, 4502c can comprise a bend between a proximal portion of the support posts 4502a, 4502b, 4502c (i.e., the portion of the support posts 4502a, 4502b, 4502c attached to the body 4504) and a distal portion of the support posts 4502a, 4502b, 4502c. In some configurations, the bends of each of the support posts 4502a, 4502b, 4502c can be at different heights h₁, h₂, h₃ in relation to the base of the support post 4502a, 4502b, 4502c and/or can have different angles Θ₁, Θ₂, Θ₃ to control the rate and/or order of the expansion of the shield 4700. For example, the first support post 4502a can include a bend with a first angle Θ₁ at a first height hi above the body 4504, the second support post 4502b can include a bend with a second angle Θ₂ at a second height h₂ above the body 4504, and the third support post 4502c can include a bend with a third angle Θ₃ at a third height h₃ above the body 4504. In some configurations, the first height hi may be greater than the second height h₂ but less than the third height h₃. In this configuration, during deployment (e.g., FIG. 42B), the second support post 4502b can extend radially outward before the first and third support posts 4502a, 4502c, and the first support posts 4502a can extend radially outward before the third support post 4502c. In some configurations, the bends of the support posts 4502a, 4502b, 4502c may all be at the same height h₁, h₂, h₃ and/or may have the same angles Θ₁, Θ₂, Θ₃. In some configurations, the bend of at least one support post 4502a, 4502b, 4502 may be at a different height h₁, h₂, h₃ and/or may have a different angle Θ₁, Θ₂, Θ₃ than the other support posts 4502a, 4502b, 4502c.

In some configurations, the difference between any two heights h₁, h₂, h₃ of the bends of the support posts 4502a, 4502b, 4502c can be approximately 2 mm, or between about 1 mm and about 10 mm. In some configurations, the angles Θ₁, Θ₂, Θ₃ of the bends of the support posts 4502a, 4502b, 4502c can be approximately 150 degrees, or between about 90 degrees and about 180 degrees.

FIGS. 42A-42B illustrate the support 4500 being deployed from a catheter 4600 or other delivery device. As shown in FIG. 42B, the support 4500 can be compressed within the catheter 4600. Once the plurality of support posts 4502 are fully deployed from the catheter 4600, as shown in FIG. 42A, the plurality of support posts 4502 can expand to form the different bends with the different angles Θ₁, Θ₂, Θ₃ at the different heights h₁, h₂, h₃.

FIGS. 43A-43C illustrate a method of deploying different embodiments of a shield 4700, 4700′ that can be attached to the support 4500 shown in FIGS. 41A-42B. FIGS. 43B-43C illustrate a shield 4700 comprising two lobes 4702, 4704 that can be attached to the support posts 4502a, 4502b, 4502c. As shown in FIG. 43C, the first and third support posts 4502a, 4502c can be attached to each of the lobes 4702, 4704 and the second support post 4502b can be attached to the hingepoint between the two lobes 4702, 4704. In some configurations, the support posts 4502 can assist the expansion of the two lobes 4703, 4704 of the shield 4700 once the shield 4700 is deployed from the catheter 4600. For example, as shown in FIG. 43B, the shield 4700 can be deployed from the catheter 4600 and expand in a first plane (e.g., along a longitudinal axis of the catheter 4600). As shown in FIG. 43C, the plurality of support posts 4502 can cause the shield 4700 to further expand in a second plane (e.g., 90 degrees from the first plane). Although the shield 4700 is shown with two lobes 4702, 4704, the shield 4700 can comprise one lobe or three or more lobes (e.g., FIG. 43A illustrates a shield 4700′ comprising a single lobe that can be attached to a single support post 4502). Additionally, as described above, the support 4500 of the shield 4700 can include a single support post 4502 (e.g., FIG. 43A) or more than three support posts 4502. Each of the lobes and support posts can have different preferred angles of separation between them or relative to a longitudinal axis of the body 4504 of the support 4500 at the desired in situ final state.

In some configurations, the shield 4700 can comprise a single wire form support that can be continuous across the top or distal side, or continuous across the bottom or proximal side of the shield. In some configurations, a shape set tube can be used to help control the position and allow for adjustment of the relative lengths of a wire advanced into each lobe of the shield 4700 to ensure proper apposition to the tissue. In some configurations, the shape set tube can comprise laser cut holes that can establish a desired angle offset of the plane of one lobe surface relative to the other.

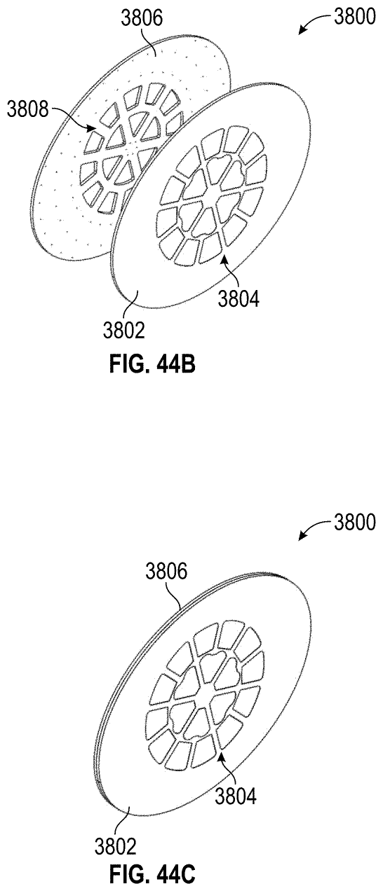

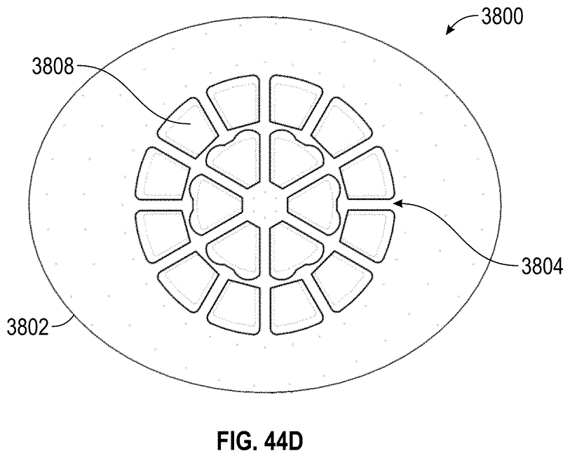

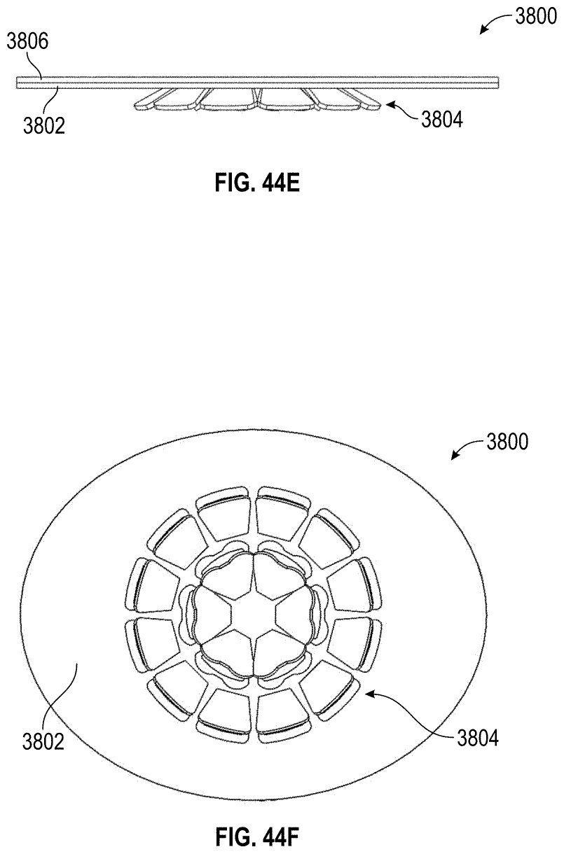

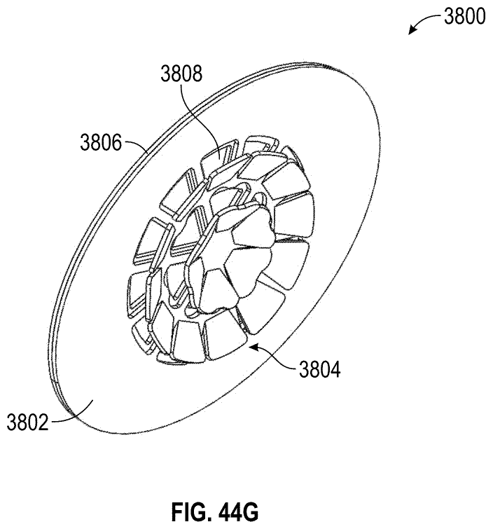

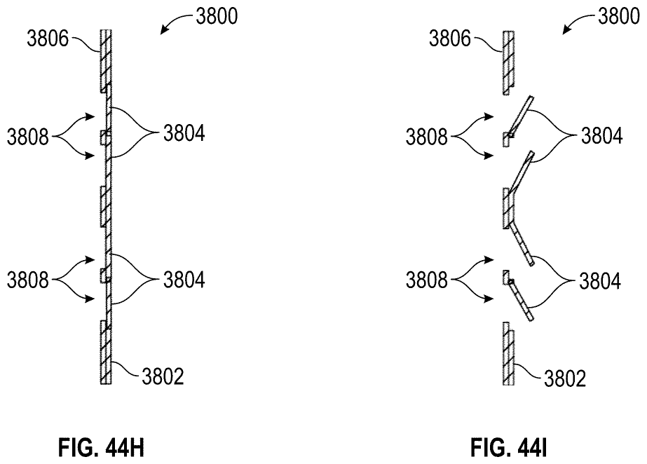

FIGS. 44A-44I illustrate another configuration of a shield 3800. The shield 3800 can include a porous backing plate or layer 3806 (e.g., FIG. 44B) that comprises a plurality of apertures 3808 and a valve plate or layer 3802 comprising a plurality of flaps 3804. The plurality of apertures 3808 can be positioned at or near the center of the porous backing plate 3806 and the plurality of flaps 3804 can be positioned at or near the center of the valve plate 3802. The plurality of apertures 3808 can comprise inner and outer apertures 3808 such that the outer apertures are radially outward from the inner apertures. Additionally, the plurality of flaps 3804 can comprise inner and outer flaps 3804 such that the outer flaps are radially outward from the inner flaps. When the shield 3800 is assembled, the plurality of apertures 3808 of the backing plate 3806 can align with the plurality of flaps 3804 of the valve plate 3802. The plurality of apertures 3808 can have similar shapes as the plurality of flaps 3804. In addition, the size of the plurality of apertures 3808 can be smaller than the plurality of flaps 3804 such that the plurality of flaps 3804 can open in one direction (i.e., when blood flows from direction of the backing plate 3806 to valve plate 3804).

As shown in FIGS. 44D-44I, the shield 3800 can include an open configuration (e.g., FIGS. 44E-44G and 44I) and a closed configuration (e.g., FIGS. 44D and 44H). In the open configuration, the plurality of flaps 3804 can move away from the porous backing plate 3806 to allow blood to flow through the plurality of apertures 3808 and the plurality of opened flaps 3804. In the closed configuration, the plurality of flaps 3804 can abut the backing plate 3806 such that blood is prevented from flowing through the plurality of closed flaps 3804 or the plurality of apertures 3808.

Although the illustrated configurations of the porous backing plate 3806 and the valve plate 3802 are shown to be circular, the backing plate 3806 and valve plate 3802 can comprise any other suitable shapes (e.g., similar shapes as the shield configurations shown in FIGS. 22-28D and 32-37). In some configurations, the plurality of apertures 3808 can be offset from the plurality of flaps 3804 such that blood can flow through the apertures 3808 and plurality of opened flaps 3804 in one direction (e.g., when blood flows from direction of the backing plate 3806 to the valve plate 3804) and is prevented from flowing through the shield 3800 in the opposite direction (e.g., when blood flows from direction of the valve plate 3804 to the backing plate 3806). In some configurations, the plurality of apertures 3808 can have different shapes than the plurality of flaps 3804. In some configurations, the backing plate 3806 can include a plurality of smaller holes configured to receive a suture for future surgical intervention and/or promote tissue ingrowth. In some configurations, the plurality of smaller holes can be used to affix the backing plate 3806 to valve plate 3802 (e.g., with sutures).

### Other Variations and Terminology

Features, materials, characteristics, or groups described in conjunction with a particular aspect, embodiment, or example are to be understood to be applicable to any other aspect, embodiment or example described herein unless incompatible therewith. All of the features disclosed in this specification (including any accompanying claims, abstract and drawings), or all of the steps of any method or process so disclosed, may be combined in any combination, except combinations where at least some of such features or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

While certain embodiments have been described, these embodiments have been presented by way of example only, and are not intended to limit the scope of protection. Indeed, the novel methods and systems described herein may be embodied in a variety of other forms. Furthermore, various omissions, substitutions and changes in the form of the methods and systems described herein may be made. Those skilled in the art will appreciate that in some embodiments, the actual steps taken in the processes illustrated or disclosed may differ from those shown in the figures. Depending on the embodiment, certain of the steps described above may be removed, others may be added. For example, the actual steps or order of steps taken in the disclosed processes may differ from those shown in the figure. Depending on the embodiment, certain of the steps described above may be removed, others may be added. Furthermore, the features and attributes of the specific embodiments disclosed above may be combined in different ways to form additional embodiments, all of which fall within the scope of the present disclosure.

Although the present disclosure includes certain embodiments, examples and applications, it will be understood by those skilled in the art that the present disclosure extends beyond the specifically disclosed embodiments to other alternative embodiments or uses and obvious modifications and equivalents thereof, including embodiments which do not provide all of the features and advantages set forth herein. Accordingly, the scope of the present disclosure is not intended to be limited by the described embodiments, and may be defined by claims as presented herein or as presented in the future.

Conditional language, such as “can,” “could,” “might,” or “may,” unless specifically stated otherwise, or otherwise understood within the context as used, is generally intended to convey that certain embodiments include, while other embodiments do not include, certain features, elements, or steps. Thus, such conditional language is not generally intended to imply that features, elements, or steps are in any way required for one or more embodiments or that one or more embodiments necessarily include logic for deciding, with or without user input or prompting, whether these features, elements, or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Likewise the term “and/or” in reference to a list of two or more items, covers all of the following interpretations of the word: any one of the items in the list, all of the items in the list, and any combination of the items in the list. Further, the term “each,” as used herein, in addition to having its ordinary meaning, can mean any subset of a set of elements to which the term “each” is applied. Additionally, the words “herein,” “above,” “below,” and words of similar import, when used in this application, refer to this application as a whole and not to any particular portions of this application.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

What is claimed is:

1. A system for isolating pulmonary pressure from left atrial pressure and/or improving cardiac output, comprising:
an intravascular shield sized and configured to be positioned in a pulmonary vein or a left atrium to restrict fluid flow from the left atrium through one or more pulmonary veins to the lungs while allowing fluid flow from the lungs through the one or more pulmonary veins to the left atrium; and
a trans-septal delivery sheath configured to contain the intravascular shield in a collapsed configuration and deliver the intravascular shield to the left atrium.

2. The system of claim 1, further comprising a pressurizing element configured to be positioned in the left atrium.

3. The system of claim 2, wherein the pressurizing element is configured to be delivered through the trans-septal delivery sheath to the left atrium.

4. The system of claim 3, wherein the intravascular shield is placed distal to the pressurizing element within the trans-septal delivery sheath.

5. The system of any one of claims 2-4, wherein the pressurizing element is a balloon.

6. The system of any one of the preceding claims, wherein the intravascular shield is sized and configured to be positioned over one or more ostia of the one or more pulmonary veins.

7. The system of any one of the preceding claims, wherein the intravascular shield comprises a one-way valve sized and configured to be positioned over or within the pulmonary vein.

8. The system of any one of the preceding claims, wherein the intravascular shield comprises an expandable frame configured to expand within the left atrium over one or more ostia of the one or more pulmonary veins.

9. The system of any one of the preceding claims, wherein the intravascular shield comprises a two or three dimensional shape sized and configured to engage a surface of the left atrium.

10. The system of any one of the preceding claims, wherein the intravascular shield comprises an expandable structural element defining a perimeter of the intravascular shield.

11. The system of claim 10, wherein the perimeter has a shape selected from the group consisting of circular, oval, clover, butterfly, single-lobed, quatrefoil, heart, two-lobed, three-lobed and four-lobed.

12. The system of any one of the preceding claims, wherein the intravascular shield comprises a non-porous layer in a center portion and at least one blood regulating flap located around a perimeter that is configured to regulate fluid flow.

13. The system of any one of the preceding claims, wherein a perimeter of the intravascular shield comprises a shape-set wire, a laser cut sheet, or a molded material that is suitable for compression and re-expansion into a catheter.

14. The system of any one of the preceding claims, wherein the intravascular shield comprises a plurality of layers.

15. The system of claim 14, wherein the plurality of layers comprises a porous layer and a non-porous layer.

16. The system of claim 15, wherein the non-porous layer has a plurality of flaps that are configured to open away from the porous layer.

17. The system of claim 16, wherein the porous layer comprises a plurality of apertures that align with the plurality of flaps of the non-porous layer.

18. The system of claim 17, wherein the plurality of apertures comprise an inner plurality of apertures and an outer plurality of apertures positioned radially outward from the inner plurality of apertures, and wherein the plurality of flaps of the valve layer comprise an inner plurality of flaps and an outer plurality of flaps positioned radially outward from the inner plurality of flaps.

19. The system of any one of claims 17 and 18, wherein the plurality of apertures comprise a similar shape as the plurality of flaps.

20. The system of any one of claims 17-19, wherein the plurality of apertures comprise smaller dimensions than the plurality of flaps.

21. The system of any one of claims 17-20, wherein the non-porous layer comprises a closed configuration when the plurality of flaps abut the porous layer and an open configuration when the plurality of flaps move away from the porous backing layer.

22. The system of any one of claims 16-21, wherein the porous layer comprises a plurality of holes configured to receive a suture, promote tissue ingrowth, and/or secure the porous layer to the non-porous layer.

23. The system of any one of claims 14-16, wherein the plurality of layers comprises a woven or knit fabric, a plurality of polymer membranes, a metal mesh, and/or a combination thereof.

24. The system of any one of the preceding claims, further comprising an elongate delivery device having a proximal end and a distal end, wherein the intravascular shield is positioned at the distal end of the delivery device.

25. An implantable cardiac device for isolating pulmonary pressure from left atrial pressure and/or improving cardiac output, the implantable cardiac device comprising:
an intravascular shield sized and configured to be positioned in a pulmonary vein or a left atrium to restrict fluid flow from the left atrium through one or more pulmonary veins to the lungs while allowing fluid flow from the lungs through the one or more pulmonary veins to the left atrium.

26. The implantable cardiac device of claim 25, wherein the intravascular shield is sized and configured to be positioned over one or more ostia of the one or more pulmonary veins.

27. The implantable cardiac device of claim 25 or 26, wherein the intravascular shield comprises a one-way valve sized and configured to be positioned over or within the pulmonary vein.

28. The implantable cardiac device of any one of claims 25-27, wherein the intravascular shield comprises an expandable frame configured to expand within the left atrium over one or more ostia of the one or more pulmonary veins.

29. The implantable cardiac device of any one of claims 25-28, wherein the intravascular shield comprises a two or three dimensional shape sized and configured to engage a surface of the left atrium.

30. The implantable cardiac device of any one of claims 25-29, wherein the intravascular shield comprises an expandable structural element defining a perimeter of the intravascular shield.

31. The implantable cardiac device of claim 30, wherein the perimeter has a shape selected from the group consisting of circular, oval, clover, butterfly, single-lobed, quatrefoil, heart, two-lobed, three-lobed and four-lobed.

32. The implantable cardiac device of any one of claims 25-31, wherein the intravascular shield comprises a non-porous layer in a center portion and at least one blood regulating flap located around a perimeter that is configured to regulate fluid flow.

33. The implantable cardiac device of any one of claims 25-32, wherein a perimeter of the intravascular shield comprises a shape-set wire, a laser cut sheet, or a molded material that is suitable for compression and re-expansion into a catheter.

34. The implantable cardiac device of any one of claims 25-33, wherein the intravascular shield comprises a plurality of layers.

35. The implantable cardiac device of claim 34, wherein the plurality of layers comprises a porous layer and a non-porous layer.

36. The implantable cardiac device of claim 35, wherein the non-porous layer has a plurality of flaps that are configured to open away from the porous layer.

37. The implantable cardiac device of claim 36, wherein the porous layer comprises a plurality of apertures that align with the plurality of flaps of the non-porous layer.

38. The implantable cardiac device of claim 37, wherein the plurality of apertures comprise an inner plurality of apertures and an outer plurality of apertures positioned radially outward from the inner plurality of apertures, and wherein the plurality of flaps of the valve layer comprise an inner plurality of flaps and an outer plurality of flaps positioned radially outward from the inner plurality of flaps.

39. The implantable cardiac device of any one of claims 37 and 38, wherein the plurality of apertures comprise a similar shape as the plurality of flaps.

40. The implantable cardiac device of any one of claims 37-39, wherein the plurality of apertures comprise smaller dimensions than the plurality of flaps.

41. The implantable cardiac device of any one of claims 37-40, wherein the non-porous layer comprises a closed configuration when the plurality of flaps abut the porous layer and an open configuration when the plurality of flaps move away from the porous backing layer.

42. The implantable cardiac device of any one of claims 36-41, wherein the porous layer comprises a plurality of holes configured to receive a suture, promote tissue ingrowth, and/or secure the porous layer to the non-porous layer.

43. The implantable cardiac device of any one of claims 34-36, wherein the plurality of layers comprises a woven or knit fabric, a plurality of polymer membranes, a metal mesh, and/or a combination thereof.

44. The implantable cardiac device of any one of claims 35-43, further comprising an elongate delivery device having a proximal end and a distal end, wherein the intravascular shield is positioned at the distal end of the delivery device.

45. A method for isolating pulmonary pressure from left atrial pressure and/or improving cardiac output, comprising using the system of any one of claims 1-24 or the implantable cardiac device of any one of claims 25-44.

46. An intravascular shield comprising one or more features of the foregoing description.

47. An implantable cardiac device comprising one or more features of the foregoing description.

48. A system for isolating pulmonary pressure from left atrial pressure and/or improving cardiac output comprising one or more features of the foregoing description.

49. A method for isolating pulmonary pressure from left atrial pressure and/or improving cardiac output comprising one or more features of the foregoing description.
